(19) United States
(12) Patent Application Publication
(10) Pub. No.: US 2026/0102581 A1
(43) Pub. Date: Apr. 16, 2026

(54) SYSTEMS AND METHODS FOR VIRTUAL REALITY-BASED HEALTH CONDITION TREATMENT

(71) Applicant: CLARITY TECHNOLOGIES, Paris (FR)

(72) Inventors: Raphael CERTAIN, Labastide st sernin (FR); Gabriel HEADLEY, Suwanee, GA (US); Timothy NOLAN, Jr., Willington, CT (US); Carolina REIS, Paris (FR); Dennis VETU, Nice (FR); Maxime RONCERAY, Villejuif (FR)

(21) Appl. No.: 19/115,702

(22) PCT Filed: Oct. 13, 2023
(86) PCT No.: PCT/US2023/076791

§ 371 Date: Mar. 26, 2025

(30) Foreign Application Priority Data
Oct. 13, 2022 (EP) 22306555.8

(51) Int. Cl. A61M 21/00

(52) U.S. Cl. CPC A61M 21/00; A61M 2021/005

(57) ABSTRACT

Systems and methods applicable, for instance, to displaying virtual reality scenarios and sensory stimulation protocols to patients afflicted with various health conditions.

### CROSS-REFERENCE TO RELATED APPLICATIONS

This application claims priority to European Patent Application No. EP22306555, filed on Oct. 13, 2022, the contents of which are incorporated herein by reference in their entirety and for all purposes.

### FIELD OF THE INVENTION

The present disclosure relates generally to health condition treatment, and more specifically, but not exclusively, to systems and methods for utilizing virtual reality in the treatment of health conditions.

### BACKGROUND OF THE INVENTION

With 90 million deaths and 16% of global annual deaths, neurological disorders are the second leading cause of death after heart disease, and the leading cause of disability. Neurological disorders include stroke, spine injuries, epilepsy, sleep disorders, Alzheimer's disease, Parkinson's disease, dementia with Lewy bodies, Primary Progressive Aphasias (PPA), frontotemporal dementia, corticobasal syndrome, progressive supranuclear palsy, posterior cortical atrophy, and others. In these diseases, the cognitive ability of the patients is impaired and may also decline over time. As the population ages, the number of people with neurological disorders is expected to increase and become a heavy burden on society and healthcare systems.

With more than 46.8 million people worldwide, dementia is one of the most devastating neurological disorders. The number of patients is expected to increase to 74.7 million people by 2030, and to 131.5 million people by 2050. More than 60% of dementia cases are caused by Alzheimer's disease (AD). Among the neuropathophysiological features associated with AD, one can generally observe an increase in amyloid-beta protein (Aβ), hyperphosphorylation of tau protein, inflammatory activity of microglia, and dendritic tree degeneration. It has also been shown that neural oscillations, particularly theta-gamma coupling, are disrupted in AD and that these impairments manifest before any behavioral deficit.

AD is a debilitating and life-threatening neurodegenerative disorder characterized by a progressive cognitive decline that manifests as memory loss, disorientation, and confusion, leading to a gradual but marked loss of self-sufficiency. Existing treatments for individuals with Alzheimer's are both expensive and offer limited clinical benefits, highlighting the urgent need for innovative solutions.

The field of neuromodulation has witnessed rapid growth in recent years, particularly in its application for enhancing cognitive functions and addressing cognitive decline. Among the various neuromodulation methods like Transcranial Magnetic Stimulation (TMS), Transcranial Electrical Stimulation (TES), and sensory stimulation, the latter standing out for its notable safety profile.

Sensory stimulation involves the precise delivery of sensory stimuli, such as visual, auditory, or vibratory inputs, at specific frequencies to influence the firing patterns of sensory brain regions and downstream areas like the memory-centric hippocampus. What sets sensory stimulation apart is its non-invasive nature, as it does not involve the direct delivery of electric current to the brain, unlike other invasive and non-invasive neuromodulation techniques such as deep brain stimulation (DBS), TMS, and TES. In essence, while DBS, TMS, and TES rely on electrical currents to modify neuronal activity, sensory stimulation harnesses the power of specific stimuli frequencies to achieve similar outcomes, offering a safer alternative for cognitive enhancement and treatment. Currently, there is no medically validated methods and hardware used for the delivery of sensory stimulation to treat or slow down the progression of cognitive decline.

A few noninvasive brain stimulation (NIBS) methods, providing the ability to modulate neural activity without penetrating the skin, have shown to be promising alternatives to pharmacological treatments. Examples include electrical stimulation (e.g., transcranial direct current stimulation (tDCS) or transcranial alternating current stimulation (tACS)), magnetic stimulation (e.g., transcranial magnetic stimulation (TMS)), and multisensory stimulation (e.g., audio-visual stimulations (AVS)). Other invasive methods include deep brain stimulation (DBS) and optogenetics. Those methods allow triggering the activity of specific brain regions, neural populations, and brainwaves in order to modulate cognitive and motor functions.

tACS, tDCS, TMS and AVS have all shown to provide benefits against cognitive decline and neurodegenerative diseases, among which are Alzheimer's disease, Parkinson's disease, dementia with Lewy bodies, PPA, frontotemporal dementia, corticobasal syndrome, progressive supranuclear palsy, and posterior cortical atrophy. These techniques have also been shown to be promising for accelerating recovery after brain injuries like stroke. Then, noninvasive brain stimulation (NIBS) methods allow for the identification of neural-based biomarkers to deliver diagnoses and assess the cognitive and motor abilities of patients.

The central nervous system (CNS) is modulated by chemical and electrical activity. Neurons, as part of the CNS, typically fire at frequencies ranging from ˜1 Hz to ˜120 Hz. Frequency bands are classified from the lower frequency to the highest frequency and named delta, theta, alpha, beta, and gamma. They are reported to be associated with and underlying specific cognitive and motor functions. For example, the frequency range of gamma brainwaves is 30 Hz and above. Gamma bands have been shown to be involved in higher cognitive abilities including decision-making, reasoning, and memory. Studies have demonstrated that Alzheimer's patients have abnormal gamma activities compared to healthy people of the same age category. NIBS aiming at training gamma activity is one approach to treating Alzheimer's disease and related pathologies.

A few studies have shown that gamma entrainment using sensory stimulus (visual or auditory at 40 Hz) in mouse models has beneficial effects on neuropathophysiological features of Alzheimer's disease. In particular, they have indicated improvement in cognitive functions. The simultaneous delivery of visual and auditory gamma stimuli increases the effects more than a single stimulation with either modality alone. Recent findings also shown that this neurostimulation technology is safe and offers improvement of AD-related-degeneration biomarkers as well as improving sleep quality. Further still, it has been shown that sensory stimulation (either visual, auditory, or both) has the potential for enhancing human neural oscillations in the theta frequency band, and enhancing memory performance in tasks probing, for instance, associative memory. Further still, it is of interest for individuals at a higher risk of developing dementia (e.g., carriers of the APOE gene and those with people diagnosed with Mild Cognitive Impairments (MCI)) to have these kinds of sensory stimulation on a regular basis (preferably one hour per day) to prevent the emergence or slow down the evolution of the disease.

Virtual reality (VR) can involve a computer-generated simulation of a three-dimensional image or environment that can be interacted with in a seemingly real or physical way by a person using equipment such as a helmet with a screen inside. VR allows the user to navigate in a digital environment, interact with the environment, and carry out specific tasks. Typical uses of VR include gaming, communication, and education.

To the extent that stimulation approaches can be said to be conventionally used in the treatment of health conditions (e.g., neurological conditions), such uses tend to be costly and/or tend to prove lacking in terms of patient engagement. As just an example, LEDs are sometimes mounted on the back of opaque lenses of spectacles, and flashed at controlled rates. Here, a user is to close their eyes during the flashing. For at least this reason, the experience typically proves unappealing. Here also, the control of flashing of the LEDs is typically handled in a rudimentary way, such as flashing the LEDs at a set rate selectable by a user.

In view of the foregoing, a need exists for improved systems and methods for treating health conditions (e.g., neurological conditions), in an effort to overcome the aforementioned obstacles and deficiencies of conventional approaches.

### SUMMARY

In accordance with various aspects disclosed herein, there is set forth a computer-implemented method, comprising:
presenting, by a computing system via a virtual reality headset, a stimulation protocol;
presenting, by the computing system via the virtual reality headset, a virtual reality scenario;
receiving, by the computing system from one or more sensors, patient physiological data; and
adapting, by the computing system using the patient physiological data, one or more of the stimulation protocol or the virtual reality scenario.

In some embodiments of the disclosed method, the patient physiological data includes patient brain activity data.

In some embodiments of the disclosed method, the presented stimulation protocol includes one or more of visual stimuli or auditory stimuli.

In some embodiments of the disclosed method, the presented stimulation protocol includes one or more of 4 Hz stimuli, 40 Hz stimuli, combined 4 Hz/40 Hz stimuli, beta-range stimuli, or alpha-range stimuli.

In some embodiments of the disclosed method, the method further comprises:
receiving, by the computing system from one or more sensors, patient brain activity data collected during patient performance of a task that elicits a brainwave state of interest; and
determining, by the computer system, from the received brain activity data, a patient-specific frequency value corresponding to the brainwave state of interest.

In some embodiments of the disclosed method, the brainwave state of interest is beta, alpha, theta, or gamma.

In some embodiments of the disclosed method, the method further comprises:
detecting, by the computing system from the received patient physiological data, abnormal neural activity; and
stopping, by the computing system, one or more of the presented stimulation protocol or the presented virtual reality scenario.

In some embodiments of the disclosed method, the presented virtual reality scenario includes one or more of a cognitive task, a functional task, or a gamified task.

In some embodiments of the disclosed method, the method further comprises:
determining, by the computing system, a score based on patient performance of said task.

In some embodiments of the disclosed method, the presentation of the stimulation protocol and the presentation of the virtual reality scenario are performed as a combination therapy component along with application of one or more pharmaceuticals.

In some embodiments of the disclosed method, the method further comprises:
determining, by the computing system, using an eye tracking system, whether patient attention is directed to visual stimuli of the presented virtual reality scenario; and
presenting, by the computing system via the virtual reality headset, patient feedback regarding said attention determination.

In some embodiments of the disclosed method, the stimulation protocol includes visual stimuli, and said presentation of the stimulation protocol includes one or more of:
modulating presentation within a VR environment of displayed content;
modulating presentation within a VR environment of a frame, wherein the frame is situated around displayed content; or
modulating presentation of at least one object within a VR environment,
wherein said modulation is at a target frequency.

In some embodiments of the disclosed method, the method further comprises:
providing, by the computing system, remote access, wherein the remote access allows for one or more of access to patient progress reports or selection of patient treatment options.

In accordance with various aspects disclosed herein, there is set forth a system comprising:
at least one processor; and
a memory storing instructions that, when executed by the at least one processor, cause the system to perform the computer-implemented method disclosed hereinabove.

In accordance with various aspects disclosed herein, there is set forth a non-transitory computer-readable storage medium including instructions that, when executed by at least one processor of a computing system, cause the computing system to perform the computer-implemented method disclosed hereinabove.

### BRIEF DESCRIPTION OF THE DRAWINGS

FIG. 1 shows examples of visual stimulation delivery, according to various embodiments.

FIG. 2 shows an example system implementation, according to various embodiments.

FIG. 3 shows an example visual stimulation delivery approach, according to various embodiments.

FIG. 4 shows a further example visual stimulation delivery approach, according to various embodiments.

FIG. 5 shows an additional example visual stimulation delivery approach, according to various embodiments.

FIG. 6 shows an example ratio versus age depiction for various groups of individuals, according to various embodiments.

FIG. 7 shows a further example ratio versus age depiction for various groups of individuals, according to various embodiments.

FIG. 8 shows an example architectural diagram, according to various embodiments.

FIG. 9 shows an example schematic view, according to various embodiments.

FIG. 10A shows an example view of a VR headset, according to various embodiments.

FIG. 10B shows a further example view of a VR headset, according to various embodiments.

FIG. 10C shows another example view of a VR headset, according to various embodiments.

FIG. 10D shows an additional example view of a VR headset, according to various embodiments.

FIG. 10E shows a still further example view of a VR headset, according to various embodiments.

FIG. 10F shows yet another example view of a VR headset, according to various embodiments.

FIG. 10G shows yet an additional example view of a VR headset, according to various embodiments.

FIG. 10H shows a further example view of a VR headset, according to various embodiments.

FIG. 11 shows an example computer, according to various embodiments.

### DETAILED DESCRIPTION

According to various embodiments, systems and methods can employ a virtual reality (VR) environment in the treatment of a health condition. The treatment can include using sensory stimulation to stimulate the brain of a patient while the patient interacts with the VR environment. Further, in various embodiments the systems and methods can monitor the health of the patient, such as by measuring various physiological parameters of the patient. In this way, the effect of the sensory stimulation on the patient can be determined, and such sensory stimulation can be appropriately adjusted. Further, the measurement of the physiological parameters can allow for monitoring of condition (e.g., disease) evolution.

Neurological conditions to which the functionality discussed herein can be applied include neurological disabilities, chronic neurological disease, and acute neurological disorders. As just some examples, neurological conditions to which the functionality discussed herein can be applied include stroke, spinal injuries, epilepsy, sleep disorders, Alzheimer's disease, Parkinson's disease, dementia (e.g., Lewy body dementia), Primary Progressive Aphasias (PPA), frontotemporal dementia, corticobasal syndrome, progressive supranuclear palsy, and posterior cortical atrophy.

To ease discussion, functionality hereinthroughout is, in general, described in connection with neurological conditions. However, it is to be understood that such functionality can also be employed in connection with other health conditions.

A virtual reality headset can be used to present the VR environment to the patient. Further, sensors and apparatuses can be used in the measurement of the various physiological parameters of the patient. The measurement can, in various embodiments, be performed in real time.

The systems and methods can utilize a library of stimulation protocols. Further, the systems and methods can utilize a library of virtual reality scenarios. In various embodiments, the virtual reality scenarios can be adaptative scenarios.

Based on the measured physiological parameters of the patient, the systems and methods can select (e.g., in real time) one or more sensory stimulation protocols (e.g., a succession of sensory stimulation protocols) from the library of stimulation protocols. Also based on the measured physiological parameters of the patient, the systems and methods can, in various embodiments, select (e.g., in real time) one or more virtual reality scenarios from the library of virtual reality scenarios. Also, in various embodiments the noted selection of sensory stimulation protocols and/or the noted selection of virtual reality scenarios can include the use of one or more feedback loops. In this way, the sensory stimulation protocols and/or the virtual reality scenarios can be adapted depending on the response of the patient as determined by the measured physiological parameters.

A given sensory stimulation protocol can be presented to the patient via a screen of the VR headset. Such use of a VR headset screen to present the sensory stimulation protocol can yield benefits including allowing for choice of where various sensory stimulations of the protocol (e.g., rhythmic flashings of VR objects) are presented in front of the patient's eyes. Such variation of the localization of the sensory stimulations during the session can provide multiple benefits to the patient. In contrast, according to conventional approaches visual stimuli are presented by LEDs surrounding right and left eye lenses of a device, and are thus spatially fixed.

A further benefit of the use of a VR headset screen to present the sensory stimulation protocol is that doing so allows for direct modulation of sensory stimulations (e.g., rhythmic sensory stimulations) on the screen as desired. In this way, a more pleasant, engaging, and immersive patient experience can be provided. As just an illustration, a gamified task can be presented to the patient, where the patient is to follow a flickering icon with their eye gaze in order to score points. Such a gamified task can not only increase adherence of the patient to the sensory stimulation protocol (e.g., long term adherence), but can also improve the clinical benefit of the sensory stimulation protocol due to the link between level of attention to sensory stimuli and neural entrainment. Sensory stimulation can also be combined with the VR environment, which can increase the therapeutic potential of the sensory stimulation. Indeed, using the VR screen to present visual stimuli makes it possible to more efficiently mobilize the visual spectrum, as there is a better perception of the stimuli when the stimuli are presented in the axis of the visual field and not around it. As such, benefits including more effective inducement of neural entrainment (and therefore improved therapeutic benefits) can accrue. It is noted that level of entrainment can, as just an example, be interpreted as a phase alignment (e.g., a significant phase alignment) and/or an increase in power of neural activity at a stimulation frequency. As such, entrainment can serve as a measure of the efficacy of a sensory stimulation protocol, wherein level of entrainment is positively correlated with the clinical outputs. Furthermore, the stimulation can be delivered during a cognitive task. As just an example, the cognitive task can entail a face-name associative task. Here, a set of pictures of unfamiliar and/or familiar faces can be displayed in association with one or more specific names. In particular, the pictures can be displayed according to a modulation (e.g., flicker) at a frequency of interest (e.g., 40 Hz). The patient can be asked to memorize these pairs. Subsequently, the patient can be asked to recall them. Through this approach, benefits including improved clinical outcomes can accrue.

According to various embodiments, the sensory stimulations provided to the patient can be rhythmic stimulations. As such, the sensory stimulations can be presented at given frequencies rather than, say, being randomly presented. The frequencies at which sensory stimulations are provided can include, for example, gamma-corresponding frequencies in the 30-100 Hz range. As such, there is typically call that the screen of the VR headset have an appropriately high enough refresh rate. In particular, there is typically call that the screen have a refresh rate that is an integer multiple of those stimulation frequencies that are to be presented. As an illustration, where the stimulation frequencies that are to be presented include a 40 Hz visual stimulus, given the Nyquist rule 80 frames per second can be a minimum refresh rate for the screen of the VR headset. According to various embodiments, a VR headset screen refresh rate of 120 Hz or higher can be used. As just an example, a VR headset screen refresh rate of 160 Hz, 200 Hz, or 240 Hz can provide for a stable delivery of 40 Hz visual stimulation.

Conventional displays, such as TV screens and monitors, typically have refresh rates that are too low to present visual stimulations between 30-60 Hz, and that are in particular too low to present visual stimulations around 40 Hz. Certain conventional VR headsets can have displays with refresh rates in the 90 Hz-120 Hz range. However, such conventional VR headset displays typically exhibit inherent variability in their refresh rates. As such these conventional VR headset displays can prove insufficient for presenting the sensory stimulations discussed herein. This discrepancy arises because conventional VR headsets are typically designed merely for immersing users in conventional VR scenarios (e.g., gaming or educational scenarios). Because stability of refresh rates can be a relevant factor in the accurate presentation of sensory stimuli, conventional VR headsets are typically suboptimal for delivering visual stimuli discussed herein.

As just some examples, measured physiological parameters can include electroencephalogram (EEG), heart rate, pupillometry, eye movement, electrodermal conductance, body temperature, respiratory rate, and heart rate variability (HRV). In various embodiments, the measured physiological parameters can further include other physiological parameters that can be collected non-invasively using appropriate sensors, and that provide (e.g., in real time) information on the physiological state of the patient.

Where the measured physiological parameters of the patient include brain activity, employed sensors can include EEG headsets and EEG electrodes. Such detection of brain activity using EEG headsets and EEG electrodes can involve measurement of electrical activity and subsequent processing so as to determine which portions of a patient's brain are active (e.g., including consideration of where sensors are placed relative to the skin of the patient). In various embodiments, a mental state of the patient can subsequently be determined. Further, in various embodiments brain activity can be measured using Functional Near-Infrared Spectroscopy (fNIRS). Here, instead of monitoring electrical neural activity directly, cortical hemodynamic activity that occurs in response to neural activity can be estimated.

Measurement of brain activity, such as via EEG, can include measurement of brainwave phase and/or the amplitude. Subsequently, a sensory stimulation protocol can be selected from the library of stimulation protocols so as to provide one or more sensory stimulations (e.g., visual stimulations) that are in phase with the actual phase measured from the patient. In this way, therapeutic effect can be optimized. As such, according to various embodiments functionality can include selection of a stimulation protocol based on measured physiological parameters, and adaption of a stimulation protocol based on measured physiological parameters. As an example, an initial task can be assigned to the patient to determine their patient-specific gamma frequency. As is discussed in greater detail hereinbelow, the task can involve the presentation of visual grating stimuli. Subsequently, stimulation can be delivered at the determined patient-specific gamma frequency.

According to various embodiments, the VR headset can include audio speakers (e.g., integrated audio speakers). The audio speakers can be used to deliver auditory sensory stimulation to the patient. The audio speakers can also be used to facilitate human-device interaction. In this regard, the audio speakers can be used to provide sounds (e.g., as instructions and/or questions) embedded in a virtual reality scenario presented by the VR headset. In this way the systems and methods discussed herein can interact with the patient, and can encourage the patient to act in the presented virtual reality scenario.

According to various embodiments, the sensory stimulation presented to the patient can include either or both of visual stimuli and auditory stimuli. Further, the auditory stimuli and the visual stimuli can be synchronized. As an example, the presentation can maintain phase-lock between the visual and auditory stimuli over time. Such synchronization can be dictated by a determined phase of monitored brainwaves (e.g., theta and/or gamma brainwaves) of the patient.

As referenced, phase-lock functionality can enable synchronization between auditory and visual stimuli. Also, phase-lock functionality can be employed where sensory stimulations having different frequencies are presented to the patient. As just an illustration, visual (or auditory) stimuli at 4 Hz can be presented along with visual (or auditory) stimuli at 40 Hz. Continuing with the illustration phase-lock functionality can be used to synchronize the 4 Hz stimuli with the 40 Hz stimuli. As a further example of phase-lock functionality, presented sensory stimulations can be phase-locked with measured brainwaves of the patient (e.g., delivery of 40 Hz visual stimuli locked to a particular phase of sensed theta waves in the auditory cortex). Also, presented sensory stimulations can be synchronized in terms of amplitude with measured brainwaves of the patient. Accordingly, synchronization that can improve the therapeutic effects of the VR headset-based treatment (e.g., cognition) can be realized.

As noted, sensory stimulation (e.g., visual stimulation) can be provided to the patient. Further to this, peripheral nerve stimulation and deep brain stimulation can be provided to the patient. The peripheral nerve stimulation and deep brain stimulation can be provided using electrodes. For example, in the case of peripheral nerve stimulation, electrodes can be placed on the surface of the patient's skin. Here, nerves that can be stimulated include the vagus nerve in the neck, the median nerve in the forearm and wrist, and the radial nerve in the forearm and wrist. In some embodiments, the virtual reality headset system can include an electrical stimulator and electrodes that can be used to provide peripheral nerve or deep brain stimulation to the patient.

Providing deep brain stimulation to the patient, such as in conjunction with providing sensory stimulation to the patient, can prove beneficial in the treatment of various neurodegenerative disorders (e.g., Parkinson's disease, essential tremor, and Alzheimer's disease), and in the treatment of various mental health disorders (e.g., obsessive-compulsive disorder and major depression). Such therapeutical combination of invasive (deep brain stimulation) and non-invasive (sensory stimulation) neuromodulation modalities can be useful in the case of Parkinson's disease, where deep electrodes can be implanted in the subthalamic nucleus, and in other brain regions of the motor network.

Peripheral nerve stimulation and deep brain stimulation both involve electrical stimulation. The peripheral nerve stimulation and deep brain stimulation can be rhythmic, such as synchronized with a frequency at which sensory stimulation is delivered by the VR headset. Alternately or additionally, peripheral nerve stimulation and/or deep brain stimulation can be delivered in synchronization with a virtual reality scenario presented by the VR headset, and delivered to assist the patient when performing a task in the virtual reality environment.

The library of stimulation protocols (e.g., a digital library or database) can include sensory stimulation protocols to provide sensory stimulations to the patient. The library of stimulation protocols can also include peripheral nerve stimulation protocols to provide peripheral nerve stimulations to the patient, and deep brain stimulation protocols to provide deep brain stimulations to the patient. The sensory stimulation protocols can specify sensory stimuli including visual stimuli and auditory stimuli. The peripheral nerve stimulation protocols and deep brain stimulation protocols can specify electric stimuli, The visual stimuli can be at specific frequencies (or at specific multiple frequencies), such as between 1 Hz and 80 Hz, between 1 Hz and 100 Hz, or at 4 Hz and/or 40 Hz. The auditory stimuli can be phase locked with the visual stimuli. The electric stimuli can include electric stimuli for application to the peripheral nerves.

The library of stimulation protocols can include stimulation protocols that can be used in the treatment and/or prevention of cognitive decline. As an example, such a stimulation protocol can start a session with visual (and/or auditory) stimuli at 4 Hz, 40 Hz, or both. The stimulation protocol can subsequently adapt (e.g., in real time) the frequency (or frequencies) of these stimuli so as to have them become in phase with measured brainwave frequencies of the patient (e.g., as measured via EEG). The adapted frequencies can be similar to the starting frequencies. As an example, the starting frequency of 4 Hz can be adapted to between 3.8 and 4.2 Hz. As another example, the starting frequency of 40 Hz can be adapted to between 38 and 42 Hz.

The frequencies of theta and gamma brainwaves of healthy people are around 4-8 Hz and 30-100 Hz, respectively. Alzheimer's patients can exhibit a reduction in brainwaves at or near 40 Hz. Further, Alzheimer's patients can exhibit a reduction in brainwaves at or near 4 Hz. And, Alzheimer's patients can exhibit a reduction in 4 Hz-40 Hz brainwave coupling. Some or all of these three aspects are suspected to be responsible (or partly responsible) for the poor memory abilities exhibited by Alzheimer's patients. As such, it can be of interest to stimulate at these frequencies. In this regard, as referenced, the library of stimulation protocols can include stimulation protocols that provide visual stimuli both at 4 Hz at 40 Hz. Further, auditory stimuli can be synchronized (e.g., phase-locked) with these visual stimuli. For instance, the auditory stimuli can be applied together with the visual stimuli from the start of the session.

The library of virtual reality scenarios (e.g., a digital library or database) can include virtual reality scenarios for presentation to the patient. The virtual reality scenarios can, as just some examples, provide immersion in a 3D virtual visual universe, a sound environment (e.g., including music), and/or animated images. In various embodiments, it can be beneficial to have the virtual reality scenarios of the library of virtual reality scenarios include scenarios that reproduce of places and spaces that are known (or potentially known) by the patient. Here, the resultant familiarity of the VR environment can allow for improved patient outcomes.

The virtual reality scenarios of the library of virtual reality scenarios, as just some examples, can allow the patient to practice relaxation exercises, can improve abilities of the patient, and/or can allow the patient to interact with the presented virtual reality universe. Such improvement of abilities of the patient can include improvement in cognitive abilities (e.g., regarding memory, attention, and/or sleep), and improvement in functional abilities (e.g., motor abilities). As an example, the improvement in cognitive abilities can be achieved by presenting cognitive games to the patient via the VR scenarios. Further, VR scenarios of the library of VR scenarios can include VR scenarios that request a patient to move in a fashion that improves motor ability of the patient. Such kinds of VR scenarios can be useful, for instance, in the treatment of stroke, and in the treatment of Parkinson's disease.

A user interface can be provided. The user interface can allow for selection of a stimulation protocol. The user interface can also allow for selection of a VR scenario (or sequence of VR scenarios), providing a personalized experience that can potentially lead to increased treatment adherence. According to various embodiments, a selected stimulation protocol or a selected VR scenario can change during a session. For example, such change can occur based on responses of the patient to the stimulation (e.g., as measured by sensors of the VR headset). As another example, such change can occur based on interactions of the patient with the presented VR environment. As further example, such change can occur based on system algorithms (e.g., algorithms that select optimal stimulation protocols and/or VR scenarios). As such some examples, stimulation changes can include changes in shape, onset, frequency, intensity lux, color, sound, and volume. Then, as such some examples, VR scenario changes can include changes in VR colors, VR shapes, VR luminance, and VR tasks.

It is noted that, in various embodiments, multiple layers can be used when presenting stimulation protocols and VR scenarios to the patient via the VR headset. For example, a first layer can be used to present the VR scenarios, and a second layer can be used to present the stimulation protocols. Also, it is noted that in various embodiments stimulation protocols can be provided to the patient while the patient performs a task in the VR environment. And, in other embodiments, stimulation protocols can be provided to the patient while the patient passively watches the VR environment.

The system can also include one or more databases (or other storage locations) that hold patient-specific information. Such patient-specific information can indicate the stimulation protocols and VR scenarios presented to given patients, and the responses of these patients to those stimulation protocols and VR scenarios. The stored responses can include physiological activity measurements as recorded by the sensors, health scores, and brain activity recordings. The patient-specific data can also include patient-specific IDs and time stamps. The databases can be accessible via the user interface of the system, and/or remotely by medical teams. Such access by medical teams can allow the teams to personalize therapeutic protocols, such as the nature of an initial stimulation protocol that is to be applied for the next session of a given patient. The system can also include one or more software modules that transmit (e.g., using encrypted transmission) the patient-specific information to a remote server or database via a computer network. In this way, medical team access to the patient-specific information can be facilitated.

The system can include at least one server on which the library of stimulation protocols and library of VR scenarios are loaded. The at least one server can be connected to control equipment via a wired or wireless communication link (e.g., an encrypted communication link). As just some examples, the communication link can employ WIFI, 4G, and/or 5G. Further, updates to the system (e.g., updates to the libraries) can be provided.

The system can work in a feedback-based manner. Here, for example, physiological parameters can be monitored and recorded, and the stimulation protocols and/or VR scenarios can be adapted (e.g., in real-time) in response thereto. In other embodiments, the system can work in a manner that does not utilize such feedback.

The functionality discussed herein (e.g., regarding the selection and presentation of stimulation protocols and VR scenarios, and regarding the analysis of physiological parameters received from the sensors) can be implemented via computer programs comprising program code instructions for performing such functionality via a computer.

According to various embodiments, administration of a stimulation protocol or VR scenario can be stopped if abnormal neural activity patterns (e.g., epileptic patterns) are identified from the received physiological parameters that are measured by the sensors. Various measured physiological parameters can be taken into consideration such identification of abnormal neural activity patterns. In this way, action can be taken to prevent the onset of adverse events (e.g., seizures) potentially arising from administered stimulation protocols or VR scenarios.

In various embodiments, on one hand the stimulation protocol can be immediately stopped. And, on the other hand, presentation of the VR scenario can be gently stopped. In this way the VR scenario can be terminated without bringing confusion to the patient. Such gentle stopping of the VR scenario can include presentation of an “urgency” scenario that provides VR imagery and/or sounds that serve to lessen confusion for the patient. Further, in various embodiments subsequent to stopping a stimulation protocol and/or a VR scenario in response to detection of abnormal neural activity patterns, the system can select a new stimulation protocol and/or VR scenario.

Further, stress state alert functionality can be provided. As discussed herein, a VR headset can be used to treat neurological disorders in a non-invasive manner. Various patients using this VR headset (e.g., patients afflicted with Alzheimer's disease) can predominately be elderly people (e.g., greater than 65 years of age). Such elderly individuals, at the current date might not be as comfortable in maneuvering digital technologies as younger generations. Also, due to the immersive sensation that one experiences when using a VR headset, it can be important to ensure that the patient is comfortable and feels safe while using the device, and that they can stop the therapy whenever desired. Moreover, as a VR headset necessarily covers a large portion of the face of the patient, it can be difficult for caregivers and medical team members to assess whether or not the patient is comfortable.

As such, the system can perform checks (e.g., recurrent checks) on the stress state of the patient. In this way, the system can send an alert signal to a caregiver or to a medical team member where a stress state is detected. The alert signal can indicate that the patient might be uncomfortable, and that the caregiver or medical team member should take action such as removing the headset or asking the patient whether they require assistance. As just some examples, the alert signal can be provided to the caregiver or medical team member via a notification sent to their smartphone, in the form of a light located on the external part of the VR headset (e.g., with the color changing based on the state of the patient), and/or via a sound played through an external speaker of the VR headset. In various embodiments, further to such alert signal to the caregiver or medical team member, the patient can be contacted within the VR environment. As just some examples, such contact can regard one or more of: a) displaying text and/or playing audio so as to ask the patient whether they are okay; b) text or audio reminding the patient that they can take off the VR headset if needed; and c) presenting a breathing exercise (or other calming VR scenario) to the patient. The system can take into account one or more of heart rate data (e.g., regarding average data and variability), pupillometry data, galvanic skin response data, and EEG data in order to ascertain whether or not the patient is experiencing a stress state.

It is reminded that an epileptic seizure is a temporary event of symptoms due to synchronization of abnormally excessive activities of neurons in the brain. “Epileptiform abnormalities” or “epilepsy waves” can be detected on an EEG. They can look like spikes, sharp waves, and spike-and-wave discharges. A seizure, on an EEG recording, can present as an appearance of abnormal discharges in bursts, which are called ictal epileptiform discharges. These discharges can increase in frequency, and lead to rapid continuous spikes and waves that progress to numerous spikes with buried waves at the peak of seizure activity. Subsequently, the waves can reappear and progressively reduce in frequency as the seizure subsides.

Detection of an epileptic seizure can be performed by the system, for instance via an analysis and calculation module thereof. As an example, the system can compare received EEG data to a system library containing representative patterns of waves during epileptic events. As another example, the system can act to detect EEG wave changes (e.g., detecting ictal epileptiform discharges, and increases in the frequency thereof). As a further example, the system can utilize machine learning models that have been trained on EEG data of patients experiencing epileptic crisis and on EEG data of patients not experiencing epileptic crisis. The EEG data of patients experiencing epileptic crisis can include EEG patterns corresponding to epileptic crisis commencement.

According to various embodiments, the system can, utilizing parameters received from the sensors, calculate diagnostic scores. These diagnostic scores can include cognitive function scores (e.g., sleep scores, memory scores, attention scores, and motor ability scores), disease risk scores, and brain health scores. The scores can be made available to medical teams, such as in the manner discussed above.

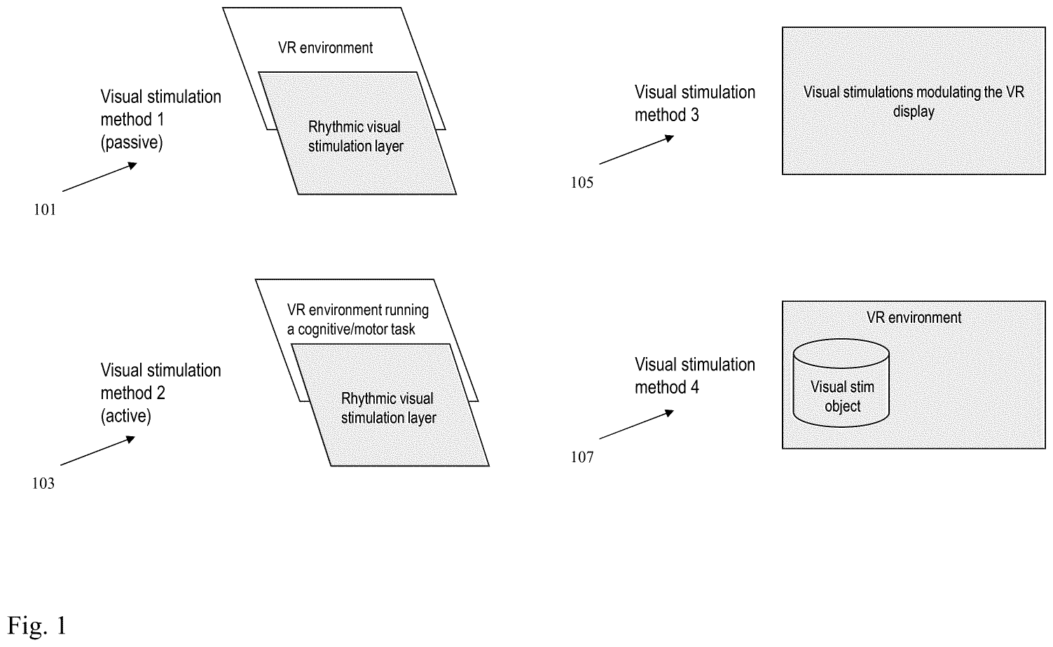

The stimulations can be delivered and displayed on the screen of the headset. With reference to FIG. 1, as just some examples, implementation can involve: a) delivering visual stimulation according to a passive approach by overlapping a stimulation layer while the patient passively watches the VR environment (101); b) delivering visual stimulation according to an active approach by overlapping a stimulation layer while the system runs a cognitive or motor task and the patient performs the task (103); c) delivering visual stimulations by modulating the appearance of a displayed video or image (and/or the entire VR environment) (105); and d) delivering visual stimulations by modulating the appearance of an object incorporated into the VR environment (107).

With further regard to visual stimuli, the visual stimuli can be generated by the system using sine waves and sinusoidal modulation (e.g., pure sinusoidal modulation) of luminance level. As just an illustration, the luminance level can be modulated from 0% luminance (e.g., full black) to 100 % luminance (e.g., full white) with a 50 % duty cycle. In some embodiments, a color other than white can be used. According to this 50% duty cycle, for a 4 Hz stimulation the luminance level can vary from 0% to 100% in 125 ms, and from 100% to 0% in 125 ms. Also according to this 50% duty cycle, for a 40 Hz stimulation the luminance level can vary from 0% to 100% in 12.5 ms, and from 100% to 0% in 12.5 ms. Further, the visual stimuli can be generated by the system using square waves and isochrone modulation (i.e., on/off modulation) of luminance level. As just an illustration, a 50% duty cycle can be used. According to this 50% duty cycle, for a 4 Hz stimulations the luminance level can be at 100% during a first 125 ms, and at 0% during a subsequent 125 ms. Also according to this 50% duty cycle, for a 40 Hz stimulation the luminance level can be at 100% during a first 12.5 ms, and at 0% during a subsequent 12.5 ms.

Where 4 Hz-40 Hz combined stimulation is used, the 40 Hz stimuli can, as just an illustration, be nested at the peak of the 4 Hz frequencies with a 0° phase offset (i.e., phase lock). Further, where nested stimulation patterns are employed, the nesting can involve nesting same-type waves (e.g., nesting a sinusoidal wave within a sinusoidal wave), or nesting different-type waves (e.g., nesting an isochrone wave within a sinusoidal wave). As just an illustration, in the case of 4 Hz-40 Hz combined stimulation a 40 Hz isochrone wave can be nested in a 4 Hz sinusoidal wave. In some embodiments, implementation of wave nesting functionality can take into account VR headset refresh rate.

With further regard to using auditory stimuli to induce auditory entrainment, the auditory stimuli can be generated by the system using amplitude modulated (AM) sounds. In particular, such use of AM can include modulating a carrier frequency depending on the stimulation frequency. It can be beneficial to use carrier frequencies between 250 Hz and 1000 Hz with a 100 % modulation depth. Using lower carrier frequencies such as these can induce significant auditory-evoked patient responses, with such lower carrier frequencies being perceived well by patients because they are related to speech perception. As just an example, the sound volume can be set at 40-80 db. In particular, a set volume of 70 dB sound pressure level (SPL) can be an optimal sound volume for sustainable auditory perception. For combined stimulation frequencies, highest frequencies can be nested at the peak of lowest frequencies with a 0° phase offset. As an illustration, for a combined stimulation frequency of 4 Hz-40 Hz, 40 Hz frequencies can be nested at the peak of 4 Hz frequencies with a 0° phase offset.

Further to using AM modulation to induce auditory entrainment, other approaches such as frequency modulation (FM) and isochrone modulation (IM) can be used. Such an FM approach can modulate the carrier frequency (e.g., a carrier frequency in the range of 250 Hz 10 kHz). Such an IM approach can involve an on/off auditory tone presented at a system-selected frequency. In various embodiments, the system can adapt carrier frequencies and/or modulation depth. Also, in various embodiments the auditory stimuli can be generated by the system via amplitude modulation of intricate sound waves, thereby departing from the use of pure tones as discussed above. These complex waveforms can include either or both of pre-existing auditory elements such as music, and sounds newly formulated by the system.

Multisensory audio-visual stimulation can be delivered by synchronizing the visual and auditory stimuli. For example, visual stimuli at 4 Hz can be synchronized with auditory stimuli at 40 Hz. As an illustration, a 0° phase offset between the visual stimuli and auditory stimuli can be used.

With regard to frequency of stimulation, it is noted that the system can modulate stimulations at specific frequencies, such as at frequencies ranging from 1 Hz to 100 Hz. As just some examples, beneficial stimulation frequencies for Alzheimer's disease and for Mild Cognitive Impairments (MCI) can include: a) 4 Hz alone; b) 40 Hz alone; and c) 4 Hz and 40 Hz combined. As just some additional examples, beneficial stimulation frequencies for Parkinson's disease can include stimulations in the beta range, and beneficial stimulation frequencies for depression can include stimulations in the alpha range. Further, patient-specific frequency values in the vicinity of these frequencies can be used. As examples, the patient-specific frequency values in the vicinity of 4 Hz can be computed by the system from EEG recordings in the theta range (4 Hz±1 Hz). Likewise, the patient-specific frequency values in the vicinity of 40 Hz can be computed by the system from EEG recordings in the gamma range (40 Hz±5 Hz. Adaptation by the system of the stimulation frequency to that indicated by the analysis of EEG recordings can be done online or offline, and can be performed in connection with visual stimuli, auditory stimuli, and combined stimulation.

With regard to neurostimulation, beyond audio-visual stimulation it is noted that electrodes for delivery of neurostimulation can be present on the VR headset, or can be implanted. In this way, the system can use the electrodes to deliver neurostimulation to specific neural targets (e.g., brain, spinal cord, and peripheral nerves). The electrodes can be connected to a power source that can deliver specific patterns of pulses. These stimulation patterns can, in various embodiments, be optimized according to data measured by the sensors (e.g., measured brainwave phase and/or amplitude data), so as to increase therapeutic effects.

A session can commence with selection of an initial sensory stimulation protocol, and selection of an initial virtual reality scenario (or series of virtual reality scenarios) to be displayed on the VR headset during the session. The selection can, as just an example, be performed using a control panel of the VR headset. The selection can be performed by the patient (if autonomous enough), by a caretaker, or by a medical team member. As just some illustrations, the initial sensory stimulation can include delivery of visual stimulation at 4 Hz, delivery of visual stimulation at 40 Hz, or delivery of visual stimulation at both 4 Hz and 40 Hz. The initial sensory stimulation protocol can include providing both visual stimulation and auditory stimulation, with the auditory stimulation phase-locked with the visual stimulation.

As an example, a session can last from about 30 minutes to about one hour, unless prematurely stopped such as in the case of detection of abnormal brainwave activity (e.g., as indicated by an epileptic EEG pattern), or at the request of the patient, the caretaker, or a medical team member. In various embodiments a 1-hour session can be considered beneficial. Also, in various embodiments daily repetition of such sessions can be considered beneficial.

The initial sensory stimulation protocol and the initial VR scenario can be altered based on analysis by the system of measured physiological parameters of the patient. For example, the system can consider brainwave phase, and the alteration can adjust the frequency of the visual stimuli (and/or of the auditory stimuli) to achieve synchronization wherein the stimulations are in phase with the brainwaves of the patient.

As referenced, the system can measure brainwave phase. Further, the system can measure brainwave amplitude. Subsequently, the system can alter the amplitude of the stimulations based on the measured brainwave amplitudes. For example, the intensity and/or the color of visual stimuli can be modified according to the amplitude of the brainwaves. In particular, the intensity can be decreased or increased, and the color can be changed to be a given color of the spectrum, or to a mix of colors. Considering auditory stimuli, as just some examples the intensity (i.e., volume) and/or frequency (i.e., pitch) of the auditory stimuli can be adjusted according to the measured brainwave amplitudes. In this way, benefits including generating auditory stimuli that result in increased brainwave amplitude can accrue.

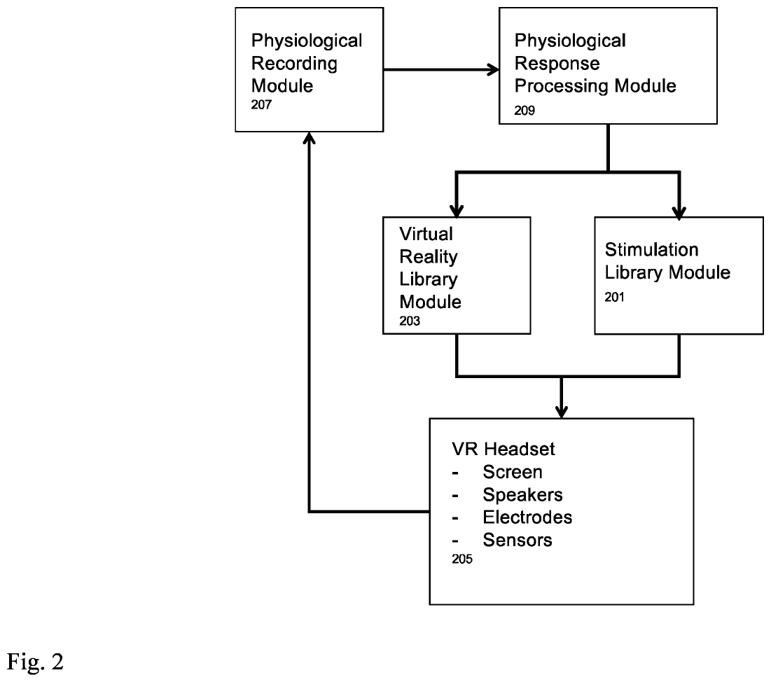

With reference to FIG. 2, the system can select a stimulation protocol from the library of stimulation protocols 201. Further, the system can select a VR scenario from the library of VR scenarios 203. The system can present the selected stimulation protocol and the selected VR scenario using a VR headset 205. Here, a data link to the VR headset can be used. The VR headset can include sensors that measure the physiological response data (e.g., EEG data). Such data can be sent over a data link to the physiological recording module 207 for recording (e.g., in real time). It is noted that, as examples, the physiological recording module 207 can record raw data, or can subject data to preprocessing prior to storage.

Subsequently, the data can be analyzed by the physiological response processing module 209. In accordance with that which is discussed above, the physiological response processing module 209 can send instruction to the library of stimulation protocols 201 for modifying the stimulation protocol. Also in accordance with that which is discussed above, the physiological response processing module 209 can send instruction to the library of VR scenarios 203 for modifying the VR scenario.

The library of stimulation protocols 201 can store instructions for providing visual stimulation protocols, auditory stimulation protocols, and combined audio-visual stimulation protocols. The library of stimulation protocols 201 can also store instructions for providing peripheral nerve stimulation protocols and deep brain stimulation protocols. As such, as just some examples the library of stimulation protocols 201 can store instructions for providing vagus nerve stimulation, transcranial direct current stimulation (tDCS), and transcranial alternating current stimulation (tACS). As indicated, stimulations can be provided as rhythmic stimulations.

It is noted that in various embodiments, various of the discussed stimulation modalities can, for instance, be delivered daily (or according to a different schedule) for a given duration. Such duration can be determined depending on factors like whether stimulation is active (provided during an assigned task) or passive (not provided during an assigned task), and the underlying health status of the patient.

The library of VR scenarios 203 can store scenarios for meditation, scenarios for relaxation, and scenarios for breathing. The library of VR scenarios 203 can also store VR scenes, animated images, sounds, and music. Further still, the library of VR scenarios 203 can store cognitive tasks and motor tasks, and scenarios for games. The cognitive tasks and motor tasks can require active participation of the patient during the virtual reality immersion.

The VR headset 205 can include a screen to be placed before the eyes of the patient, and speakers to be placed close to the ears of the patient. As such, the patient can be exposed to system-selected VR scenarios.

The system can include sensors for capturing physiological data of the patient. The captured physiological data can be stored by the physiological recording module 207. As just some examples, the physiological data captured by the sensors can include EEG data, ECG (electrocardiogram) data, EOG (electrooculogram) data, PPG (photoplethysmogram) data, EMG (electromyogram) data, heart rate data, respiratory data, pupillometric data (e.g., including eye tracking data), galvanic skin response data, accelerometer data, gyroscope data, and temperature data. Various of the sensors (e.g., sensors for capturing the noted EEG data) can be included in the VR headset 205. Also, various of the sensors can be mounted on equipment such as belts, harnesses, and gloves. In this way, the sensors can be positioned at appropriate locations on the body of the patient.

As referenced, the physiological response processing module 209 can perform operations including determining (e.g., in real time) the nature and variation of the measured physiological parameters. The output of such analysis (e.g., a physiological score or a crossing of a power threshold) can be stored in a memory either remote from or present in the module 209. In this way, as just some examples: a) evolution of parameters during a session can be ascertained; b) evolution of parameters over time (e.g., parameter variation between two sessions) can be ascertained; and c) stimulation protocols and VR scenarios can be altered.

The physiological response processing module 209 can also calculate health scores (e.g., global health scores) based either on one physiological parameter or on several physiological parameters taken in combination. Further still, the physiological response processing module 209 can calculate an entrainment score. In particular, the physiological response processing module 209 can consider measured EEG data of the patient that is collected during a session in relation to a desired brainwave goal for the patient as specified by stimulations that are presented to the patient. Further, the physiological response processing module 209 can evaluate and/or score tasks assigned to the patient in the virtual reality universe. Additionally, the physiological response processing module 209 can, in accordance with that which is discussed above, inspect collected patient EEG data for abnormal neural activity patterns, and terminate a session where such an abnormal pattern is found. In various embodiments, the operation of the physiological response processing module 209 can be implemented via machine learning approaches. Further, in various embodiments the physiological response processing module 209 can quantify predominant brain frequencies of the patient.

In various embodiments, the physiological response processing module 209 can include a machine learning model (MLM) such as a reinforcement learning (RL)-based MLM. The model can receive inputs including a stimulation presented to a patient (e.g., a 40 Hz sensory stimulation), EEG data received from the patient, and a desired entrainment state for the patient (e.g., gamma entrainment). The MLM can generate as output a suggested change in the stimulation to be presented to the patient. Where implementation of the suggested stimulation change generated by the MLM results in the patient achieving (or moving closer to) the desired entrainment state, the MLM can receive a positive reward. Otherwise, the MLM can receive no reward (or a negative reward). In this way the MLM can, over time, learn to more effectively generate stimulation change suggestions.

The noted modules discussed in connection with FIG. 2 can be implemented using software and/or hardware. As such, the system of FIG. 2 can include one or more executed software modules, one or more processors (e.g., CPUs or GPUs), one or more amplifiers (e.g., EEG amplifiers), one or more communication devices (e.g., Bluetooth and/or WIFI devices), and one or more power supplies.

The system can also include at least one display screen to convey (e.g., in real time) information regarding a session. As just some examples, the displayed information can include entrainment score evolution and/or health score evolution. As further examples, the display screen can also convey (e.g., in real time) values measured by the sensors, and the degree of progress of the session (e.g., patient progress in completing virtual reality scenario tasks, specification of provided stimulations, and time elapsed during a session). As such, the displayed information can be made available to patients, caretakers, medical team members, and others. As an example, the display screen can be integrated with the system of FIG. 2. As another example, the display screen can be remote from the system of FIG. 2, such as being implemented by a computer, a cell phone, or a touch-sensitive tablet that is in communication (e.g., via a wireless link) with the system of FIG. 2.

In various embodiments, one or more algorithms can be used to discard or correct sensor-measured physiological parameters that are identifiably erroneous (e.g., outside a range considered normal for a patient). In this way, the system can prevent such physiological parameters from being further acted upon by the system (e.g., further acted upon by the physiological response processing module 209).

The functionality discussed herein can yield a multitude of benefits. As such some examples, such benefits include: a) easy home-based access and performance; b) easy remote monitoring of patient use of the system; c) facilitation of neurological disorder diagnosis (e.g., yielding time and cost reductions); and d) facilitation of the delivery of noninvasive brain stimulations at home. As just some additional examples, further benefits can include: a) increasing patient adherence to digital therapeutics by making them engaging, pleasant and funny; b) improving and accelerating a patient's path to neurological improvement (e.g., slowing neurological health deterioration); c) improving prevention, care quality, medical practices and personalization of medical guidelines based on actual daily health measurements; and d) providing targeted and individualized power precision medicine solutions for neurological disorders, while making brain health solution more accessible. Further still, the functionality discussed herein can: a) improve quality of life for patients and caregivers, while avoiding a need for advanced assistance; and b) allowing medical teams to save time in monitoring neurological patients.

Stimulation delivery will now be discussed in greater detail. When used for therapeutical purposes, neuromodulation techniques, both invasive and non-invasive, can aim to modulate neural activity at frequencies that are linked to specific disease symptoms. For example, neuromodulatory therapy including deep brain stimulation at high frequencies (e.g., >120 Hz) can disrupt the excessive beta power (e.g., 15-35 Hz) found in the motor brain regions of patients with Parkinson's disease. More generally, the frequencies at which stimulation is to be delivered in order to treat a given neurological condition can be based on neural oscillatory correlates of specific neurological conditions and symptoms.

The functionality discussed herein includes approaches for delivering audiovisual sensory stimulation with the aim of inducing neural entrainment to specific frequencies. In one aspect, the functionality discussed herein can serve to enhance neural synchronization (e.g., as evidenced by enhanced power) at frequencies that are linked to healthy cognitive performance, and that therefore are reduced in people suffering from cognitive impairment. The frequencies of interest according to the functionality discussed herein can include those in the theta and low gamma ranges (around 4-8 Hz and around 30-60 Hz, respectively), due to their role in higher cognitive functions (e.g., memory formation and sensory perception). Still, the system can, in various embodiments, support and deliver any frequency in a range of 0.1 Hz to 45 Hz. Moreover: 1) exposure (e.g., chronic exposure) of Alzheimer's patients to 40 Hz audiovisual stimulation is capable of delaying the progression of Alzheimer's disease as measured by reduced brain atrophy and preserved ability to perform daily activities; and 2) audiovisual sensory stimulation (e.g., acute audiovisual sensory stimulation) at 4 Hz during a cognitive task can improve associative memory performance. The functionality discussed herein includes intervention that delivers audiovisual stimulation through a VR headset. As just some examples, this stimulation includes stimulation at frequencies that are linked to cognitive functions, such as within the 4-8 Hz theta and 30-60 Hz gamma ranges.

According to various embodiments, two sensory stimulation protocols can be used: 1) an open loop stimulation protocol that uses non-patient-specific stimulation frequencies; and 2) a closed loop stimulation protocol that uses patient-specific stimulation frequencies.

As noted, the open loop stimulation protocol can use non-patient-specific stimulation frequencies. Said somewhat differently, the open loop stimulation protocol can use fixed stimulation frequencies. As an illustration, for the treatment of cognitive decline provided sensory stimulation can occur at pre-determined frequencies such as at 4 Hz, 40 Hz, or a complex pattern where 40 Hz is nested in a 4 Hz carrier wave. Here, the rate of modulation of visual stimuli that is presented via the VR headset can occur at such predetermined frequencies. Likewise here, the rate of modulation of presented auditory stimuli (e.g., presented via in-phase rate of volume modulation) can occur at such predetermined frequencies. Optionally, visual stimuli and auditory stimuli can assume different stimulation frequencies from each other (e.g., visual stimuli at 4 Hz and auditory stimuli at 40 Hz). In various embodiments, such two oscillations can be in-phase.

As noted, the closed loop stimulation protocol can use patient-specific stimulation frequencies. Said somewhat differently, the closed loop stimulation protocol can entail a personalization of stimulation frequency within the ranges of interest (e.g., theta and gamma). As such, the closed loop stimulation protocol can include finding patient-specific frequency values that are linked to various cognitive-related processes.

To achieve this, the system can use EEG sensors to record brain activity of the patient while the patient performs a task that elicits the brainwave state of interest (e.g., gamma state). The system (e.g., using the physiological response processing module 209) can extract from the EEG data the predominant frequencies thereof. As just some examples, the extraction can be performed using power spectral density Morlet wavelet and/or Fourier Transform approaches. In this way, the patient-specific frequency value corresponding to the brainwave state of interest can be determined. It is noted that, in various embodiments, further to determining predominant frequency, phase and amplitude can also be ascertained.

Subsequently, the determined patient-specific frequency value corresponding to the brainwave state of interest (e.g., gamma) can be used to deliver stimulation to the patient, thereby achieving neural entrainment at that brainwave state of interest.

According to various embodiments, two approaches can be used in having the patient perform a task that elicits a brainwave state of interest (e.g., theta or gamma state) while EEG sensors record brain activity of the patient, as noted. The first such approach is endogenous extraction through visual grating stimuli. The second such approach is low and high frequency sweep.

Turning to endogenous extraction through visual grating stimuli., repeated presentation of both moving and stationary grating stimulus can elicit narrow-band gamma oscillations (e.g., 30-50 Hz) in the visual cortex. A task (e.g., a short task) of repeated visual grating presentation can be presented to the patient (e.g., via the VR headset). The patient can perform the task, thereby allowing extraction of their endogenous gamma frequency. With reference to that which is discussed above, computation of the frequency value can be obtained via power spectral density and/or time-frequency analyses, as just some examples. The determined frequency value can, as referenced, be used to control the frequencies at which visual stimuli and/or auditory stimuli is delivered. Although, in the interest of illustration by way of example, discussion of using visual grating stimuli to determine patient-specific gamma frequencies is discussed, according to various embodiments such approaches can be used to determine patient-specific frequencies for other brainwave states.

Turning to low and high frequency sweep, flicker-induced brain entrainment in the visual cortices can occur up to a stimulation frequency of about 90 Hz. Moreover, the magnitude of flicker-induced entrainment can be variable across subjects and stimulation frequencies. With this in mind, the at-hand low and high frequency sweep approach can use a flickering task where the frequency of a flickering stimulus (e.g., presented via the VR headset) is changed (e.g., gradually changed) while EEG is being recorded. The range of frequencies tested can include, as just some examples: a) a low-frequency sweep around theta frequencies (e.g., from 3 Hz to 5 Hz increments of 1 Hz); and b) a high frequency sweep around gamma frequencies (e.g., from 35 Hz to 45 Hz in increments of 1 Hz). The sweeps (e.g., the theta sweep and/or the gamma sweep) are typically repeated several times so as to obtain sufficient data for the computation of appropriate parameters (e.g., power and/or phase alignment) across stimulation trials at a given frequency.

The frequency that leads to the higher level of entrainment (e.g., as evidenced by a power increase and/or phase alignment) in the theta and/or the gamma sweep can be used as the frequency at which visual stimuli and/or auditory stimuli is delivered (e.g., the rate at which the brightness of an object in the displayed VR environment is flickered). For example, if subject A shows a higher power increase and phase alignment at 42 Hz compared to ten other frequencies of stimulation (e.g., from 35 Hz to 45 Hz), then stimulation can be set to occur at 42 Hz for that specific individual. Although, in the interest of illustration by way of example, discussion of using low and high frequency sweep to determine patient-specific theta and gamma frequencies is discussed, according to various embodiments such approaches can be used to determine patient-specific frequencies for other brainwave states.

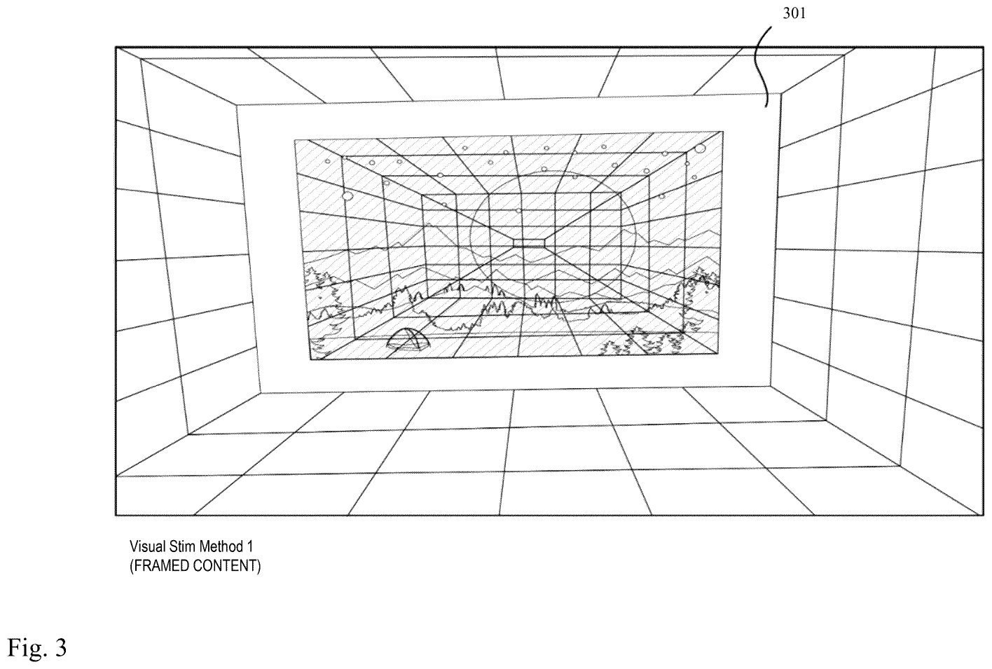

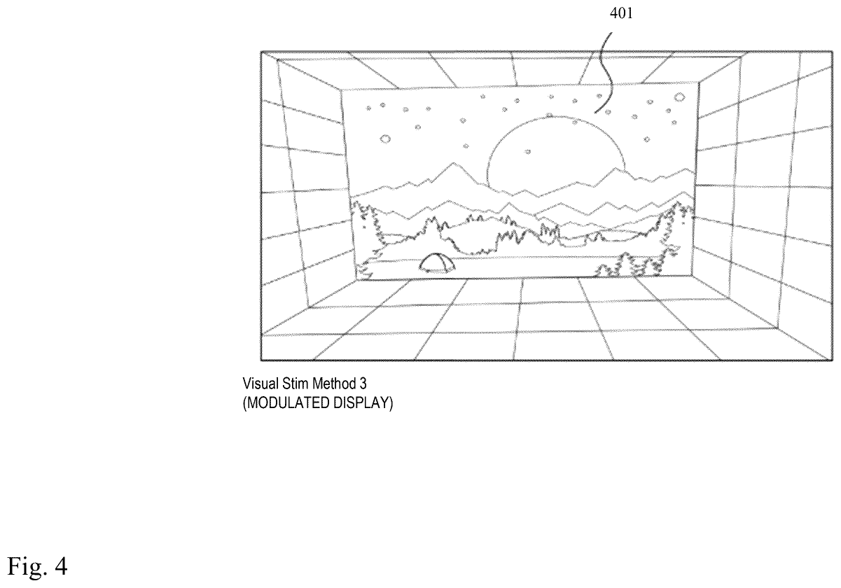

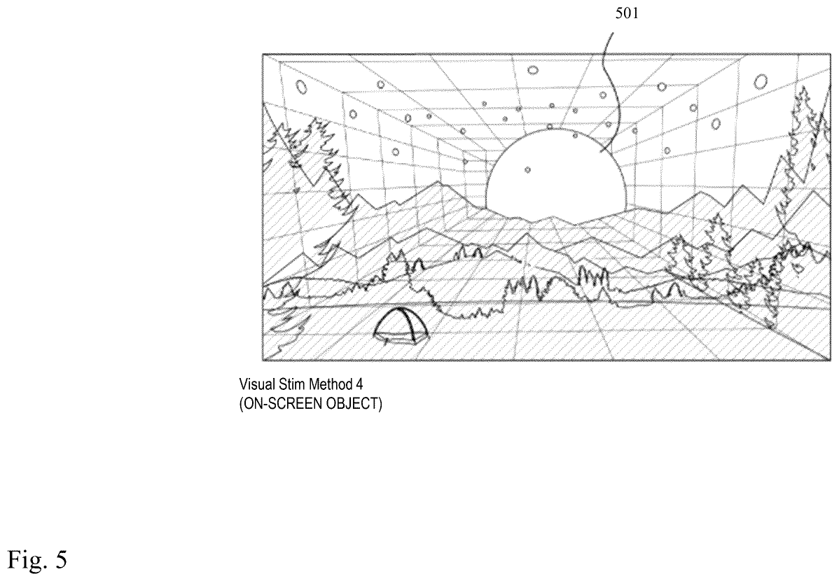

Approaches for delivering sensory stimuli via the VR headset will now be discussed in greater detail. With reference to FIG. 3, according to a first example approach visual stimulation can be delivered via a frame 301 that is situated around a video or image presented by the displayed VR environment. The frame can flicker at the target frequency. Then, with reference to FIG. 4, according to a second example approach visual stimulation can be delivered through a presented video or image itself 401 flickering at the target frequency. And, with reference to FIG. 5, according to a third example approach visual stimulation can be delivered through one or more flickering components 501 (e.g., objects within) the displayed VR environment. According to these approaches, the displayed VR environment can be an immersive 360° VR environment.

For the first example approach, the point-of-view of the patient can be placed in front of a large rectangular video screen or image that is presented by the VR environment. The size and the patient's distance from the screen can be comparable to the patient watching a film in the theater. A frame can surround the screen on all four of its sides. While the video is playing or image is displayed, the frame can flicker at the target stimulation frequency (e.g., alternating between differing values of luminosity to produce the desired visual flickering effect).

For the second example approach, the point-of-view of the patient can, like the first approach, be placed in front of a large rectangular video screen or image that is presented by the VR environment. However, unlike the first approach, there is no frame surrounding the video or image. Instead, while the video is playing or image is displayed, the video or image itself (and/or the entire VR environment) can flicker at the target frequency to deliver the stimulation. As just an example, the video or image can rapidly alternate between different values of luminosity to produce the desired visual flickering effect. In various embodiments, the flickering of the video or image can take the form of flickering a landscape of the VR environment.

For the third example approach, the point-of-view of the patient can be placed at some point within the VR environment. The VR environment can, as just an example, be calming to the patient and can provide a safe digital space to deliver the stimulation. One or more components (e.g., objects) within the VR environment (e.g., objects that are in the patient's view) can flicker at the target frequency. In this way, the desired visual stimulation can be delivered to the patient.

The content displayed to the user via the VR headset can, as just some examples, be video, image or a 360° VR environment. Moreover, the displayed content can be passive or active content. Such passive content can include content with which the patient does not need to engage, beyond perhaps paying attention to what is displayed. Such active content can include content with which the patient is to engage. As examples, such content can include cognitive, functional, and/or gamified tasks. For instance, such tasks can aim to assess or improve the cognitive or functional abilities of the patient.

As just some examples, the content, whether it is passive or active, can take the form of: 1) a natural or digital representation of landscapes (e.g., forest, beach, or space); 2) daily life places (e.g., a home, a backyard, a street, or a city); 3) an animated environment; and/or 4) personal patient memories (e.g., family pictures or family memories). For personal patient memories, the patient can, for instance, upload the corresponding content to the system using their mobile phone. Subsequently, the system can apply a VR synthesis technique to the uploaded content, so as to embed it for display in the VR environment.

Turning to auditory stimuli, according to the foregoing three example approaches presentation of auditory stimuli can include providing an audio source within the VR environment (e.g., situated at one or more locations in the VR environment). In this way, the patient can hear the auditory stimuli through the VR headset. As referenced, the presented auditory stimuli can be modulated at the target stimulation frequency. In various embodiments the visual stimuli and auditory stimuli can both be presented in accordance with the target frequency, and can be synchronized.

For sensory stimulation to successfully induce neural entrainment, on one hand the presentation rate (e.g., the rate of flickering) of the presented stimuli (e.g., visual and/or auditory stimuli) should typically be stable. And, on the other hand, patient attention should typically be focused on the stimuli. According to various embodiments, the system can act to promote such patient focus.

Turning to promoting patient focus on presented visual stimuli, the system can act to determine whether or not the patient is directing their attention to the visual stimuli. According to various embodiments, an eye tracking system can be used. The eye tracking system can be integrated into the VR headset. The eye tracking system can evaluate the level of visual attention paid by the patient to the visual stimuli (e.g., a flickering of a specific object in the VR environment). Further, feedback can be provided to the patient regarding the level of visual attention paid.

The eye tracking system can allow for determination of the coordinates of the gaze of the patient (e.g., of the exact gaze point coordinates). In this way, the system can be aware of whether or not the patient is directing their gaze to the part of the VR environment that is displaying the visual stimuli (e.g., flickering). In various embodiments, the eye tracking system can also determine whether or not the patient has their eyes open. Here, it is noted that the patient having their eyes open can be a factor of importance in assuring that the visual stimuli is perceived by the patient to a degree that can induce sufficient neural entrainment.

If application of the eye tracking system reveals that the patient has their eyes closed, as one example a message can be displayed on the screen of the VR headset, and/or audio asking the patient to open their eyes can be played. As another example, action can be taken where the eye tracking system determines that: 1) the patient has their eyes open; but 2) the eye gaze is directed to coordinates that do not correspond to those where visual stimuli is being delivered (e.g., is being delivered for a specific amount of time). The action can include presenting to the patient, within the VR environment, a visual (e.g., textual) or audio feedback message. The feedback message can direct the patient to pay attention to the visual stimuli. For instance, the feedback message can characterize the visual stimuli as “the part of the VR environment that is flickering,” or by using similar language. As another example, the action can include a change in the VR environment, and/or a change in the visual stimuli (e.g., a change in a VR object whose brightness is being modulated). As an illustration, suppose that the VR environment includes a flickering white triangle object (e.g., that is flickering at 40 Hz). Continuing with the illustration, where the patient is not directing their visual attention to the triangle, a glowing feature (or other feature) can be drawn around the triangle so as to draw the attention of the patient to the triangle. In various embodiments, continuous attention feedback (e.g., a cursor displayed on top of or one the side of the VR environment) can be presented to the patient.

Changes in alpha (e.g., 8-12 Hz) synchronization (e.g., as observed as changes in power) are understood to influence or reflect allocation of visual attention to different aspects of visual stimuli. In particular, there is an apparent inverse relationship between alpha power and visual attention. More specifically, when human attention is actively focused on a visual task, alpha power tends to decrease in the occipital and parietal cortices-the first region being responsible for the processing of visual information, and the latter playing a role in spatial processing and in directing attention.

As such, according to various embodiments where application of the eye tracking system determines that the eye gaze of the patient is directed to visual stimuli, additional operations can be performed. According to these additional operations, the system can analyze EEG data received from EEG sensors that receive signals from the noted occipital and/or parietal areas of the brain of the patient. In this way, the system can assess the level of attention deployed to the visual stimuli to which the eye gaze of the patient is directed.

For example, once the eye tracking system determines that the eye gaze of the patient is directed to given visual stimuli, occipital and/or parietal neural activity can be recorded during sensory stimulation periods. Subsequently, ascertained variation in alpha (e.g., variation in alpha power) can be used as a proxy for visual attention. For instance, using: a) a power threshold for the alpha activity recorded during stimulation periods; and/or b) a change in alpha power compared to one or more periods just before stimulation onset, the system can assess whether attention levels to the visual stimuli are satisfactory. Where the system determines that the attention levels are not satisfactory, action can be taken. For instance, within the VR environment the patient can be presented with a visual or textual message that directs the patient to pay greater attention to the visual stimuli.

Turning to promoting patient focus on presented auditory stimuli, it is understood that there is a link between pupil dilation and cognitive processes, such as auditory memory load and auditory processing demands. As such, according to various embodiments the system can utilize pupillometry approaches to measure pupil dilation during auditory stimuli presentation to assess the attention devoted to the auditory stimuli by the patient. As an example, the eye tracking system can be used to perform such measurement of pupil dilation.

The system can utilize the determined pupil dilation metric information to provide therapeutic efficacy measures. For instance, the system can measure a baseline pupil diameter for a patient when the patient is not focused on an auditory stimulus (or not focused on an auditory task). Subsequently, the system can interpret recurrence of this baseline pupil diameter (or a similar pupil diameter) as being indicative of the patient not being focused on a subsequent auditory stimulus (or auditory task). The system can consider this lack of focus as indicative of reduced task engagement, and consequently indicative of reduced predicted neural entrainment and reduced clinical benefit.

Further still, the system can, based on this lack of focus, provide feedback to the patient within the VR environment. As an example, the feedback can direct the patient back to the stimuli, such as by adding items to the stimulation protocol (e.g., a break from the stimulation environment to do a breathing task). As another example, the feedback can change the VR environment where the auditory stimulation is being delivered. In particular, the change can serve to increase patient enjoyment and motivation, and hence attention to an at-hand task. As just an illustration, within the VR environment the patient can be presented with an icon that challenges the patient to pay more attention to the auditory stimuli in order to receive greater points in a gamified stimulation task. It is noted that, in general, the discussed approaches for using pupillometry to assess the attention devoted to auditory stimuli are used for sensory stimulation protocols where only auditory stimuli are being delivered. Such holds because brightness variability corresponding to presented visual stimulation (e.g., modulated flashing) can impact pupil diameter, and therefore can contaminate the auditory-evoked pupil response approach discussed above.

As referenced, according to various embodiments different health-related scores can be collected, the scores primarily being measured digitally and non-invasively. As just some examples, such scores can include physiological measurements (e.g., heart rate and temperature), arousal state measurements (e.g., measuring saccade velocity, pupil dilation, and blink rate), motor function, and cognitive scores (e.g., measuring behavioral performance in cognitive tasks performed through the VR headset). Such measurement of motor function can involve, for example, a smartwatch that contains an accelerometer and gyroscope. Data collection from the accelerometer and gyroscope can be synchronized, for instance, with a motor task displayed in the VR headset to record tremor levels during specific movements in patients suffering from essential tremor.

The functionality discussed herein is comprehensive, not only treating neurological disorders, but also tracking disease progression and assessing treatment efficacy. It is noted that, these three aspects can be operationally linked. As an example, a cognitive task can be presented to a cognitively impaired patient along with the delivery of a 40 Hz audiovisual stimulation. Such action can serve to: 1) treat the neurological disorder of the patient; 2) collect cognitive scores of that same task over time in order to track disease progression; and 3) assess treatment efficacy (e.g., in connection with sharing corresponding scores with medical team members).

As just some examples, health scores that can be collected according to the functionality discussed herein include behavioral scores, neural scores, and scores related to tracking motor functions.

Turning to behavioral scores, such scores can, as just some examples, be obtained from the following tasks: a) free recall tasks; b) cued recall tasks; c) working memory tasks; d) spatial memory tasks; e) episodic memory tasks; f) associative memory tasks; g) semantic memory tasks; h) procedural memory tasks; i) source memory tasks; j) implicit memory tasks; k) prospective memory tasks; and l) pattern recognition tasks. Here, a decline in associative memory can be a prominent indicator of cognitive deterioration in individuals with Alzheimer's disease. An associative memory task, which typically involves the pairing of visual and audio stimuli during encoding and subsequent testing in a recall phase, can yield an associative memory score (e.g., an accuracy percentage). This score can be monitored longitudinally, thereby serving as a proxy for tracking disease progression. The trajectory of performance in the associative memory task can serve as an indicative measure of associative memory decline. When combined with other assessment scores and clinical information, it can provide a comprehensive overview of the evolving disease status of a patient. According to various embodiments, functional abilities beyond memory can be scored, with or without stimulation being provided during the scoring process.

Turning to neural scores it is noted that different neural oscillations (e.g., brain activity collected via EEG sensors at specific frequencies) can be correlated with (and in some cases causally related to) different aspects of memory formation and recall. Examples of this include: a) oscillation in the theta, alpha, and gamma bands—and also coupling among theta and gamma oscillations—during awake states; and b) slow oscillations, alpha oscillations, and ripples during sleep.

As such, the functionality discussed herein can include the longitudinal recording of neural activity during the performance of different memory tasks so as to provide for the monitoring of disease progression. Typically, such functionality is implemented in a patient-specific fashion due to the inter-subject variability observed in neural activity levels. As an example, the above-discussed technique of endogenous extraction through visual grating stimuli can be used to elicit gamma frequencies from a patient.

Gamma oscillations are implicated in a wide range of sensory and higher cognitive functions such as attention, perception, and memory formation. In particular, two deficits associated with Alzheimer's disease are reduction in the power of gamma oscillations and impairment in cognitive functions. This observation can be replicated using grating stimuli, where a reduced endogenous gamma power in cognitively impaired patients (e.g., patients suffering from mild cognitive impairment and/or Alzheimer's disease) can be found relative to age-matched controls. According to various embodiments, the level of endogenous gamma induced by visual gratings can be assessed longitudinally, in order to track the cognitive impairment evolution of patients. The techniques employed for calculating the power of endogenous gamma oscillations can include, as just some examples, power spectral density, Morlet wavelet analysis, and Fourier Transform. Further, neural scores can be formulated based on various neural signal processing methods (e.g., amplitude, power, and phase analyses) at different frequencies (e.g., theta, gamma, and the cross-frequency coupling between theta and gamma). As referenced, in various embodiments scores related to motor functions can be ascertained. Here, various oscillations can be of interest. For example, in the case of Parkinson's disease it can be of interest to track the evolution of beta and gamma oscillations during rest and motor task periods.

According to various embodiments, a theta-gamma scoring system can be employed. Theta-gamma cross-frequency coupling has been shown to be disrupted in patients with cognitive impairment due to dementia or mild cognitive impairment (MCI) compared to healthy people of the same age group. This coupling strength has also been shown to be correlated to cognitive performance, (e.g., working memory). In particular, the increase in theta or the decrease in gamma have been individually related to cognitive decline.

According to the functionality discussed herein, a theta-gamma ratio can be used as: 1) an early marker of disease; 2) a marker of risk, and/or 3) a marker of disease evolution. Taken alone or together with other biomarkers (e.g., heart rate, pupillometry, and/or proteins like amyloid-beta), the theta-gamma ratio can result in health and disease progression scores that indicate treatment efficacy. As one example, the theta-gamma ratio can be calculated by quantifying the relative power at frequency peaks both in theta and gamma ranges during rest EEG sessions. As another example, the theta-gamma ratio can be calculated based on cross-frequency phase-amplitude coupling (PAC) and phase synchronization (e.g., including power, strength, and/or onset metrics).

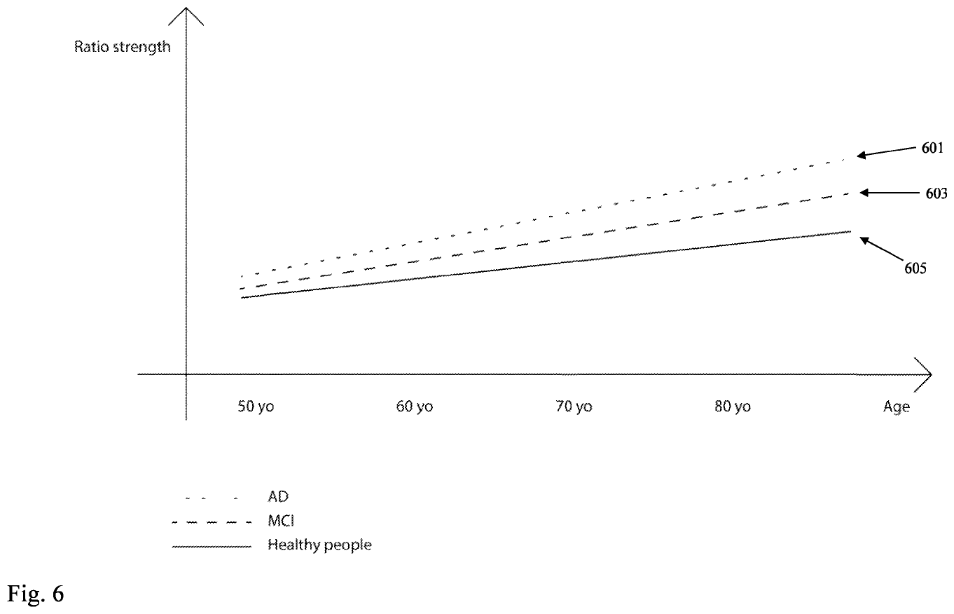

A baseline score for the ratio can be established, as just one example, by measuring and quantifying the ratio for a cohort of healthy people aged between 50 years of age and 85 years of age, and segregated into age groups with a resolution of five years. A linear assessment of the evolution of the ratio can correspond to a normal evolution of the ratio in healthy people with no markers of Alzheimer's disease or MCI. The patient's ratio can then be compared to this linear evolution. Subsequently, differences can be extracted from the comparison so as to quantify the level of progression of the disease. It is noted that although the ratio can increase naturally due to normal aging, the increase is amplified in Alzheimer's disease and MCI patients. This amplification or difference can be quantified to establish the theta-gamma score. Turning to FIG. 6, depicted is ratio versus age for Alzheimer's disease patients 601, for MCI patients 603, and for healthy people 605.

According to a first example use case, the ratio can be used as a risk-factor measure of the silent progression of MCI or Alzheimer's disease, independently or in combination with prodromal disease biomarkers (e.g., cerebrospinal fluid (CSF), genetic profiles (like APOE4), and other blood biomarkers like proteins). According to a second example use case, the ratio can be used as a biomarker, for instance as part of a clinical diagnosis along with other biomarkers. Then, according to a third example use case, the ratio can be used as a biomarker of disease progression along with a treatment protocol such as the sensory stimulation provided via VR headset as discussed herein.

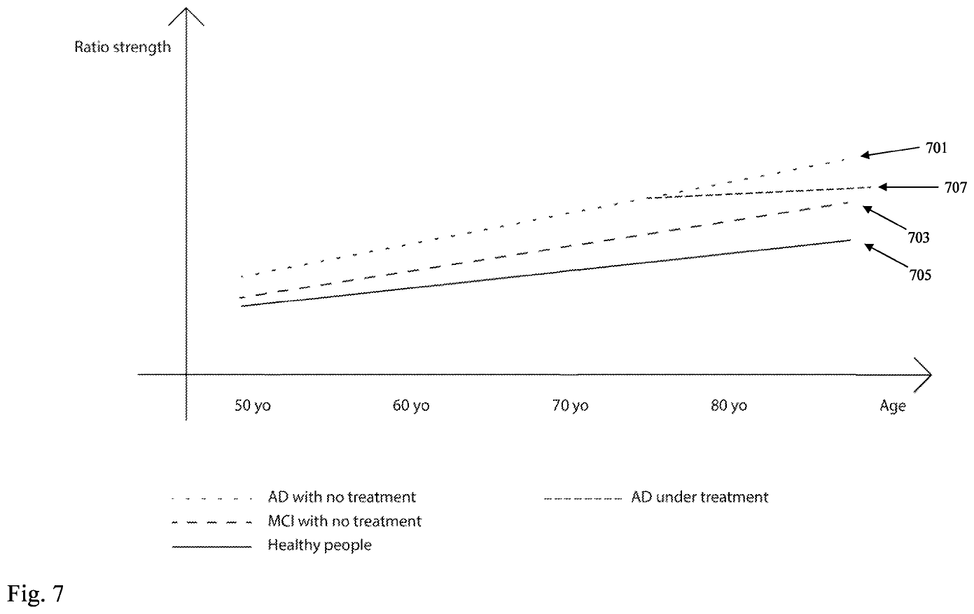

The evolution of the theta-gamma score can be established via multiple approaches. According to a first example approach, the ratio of the patient can be compared to the baseline. According to a second example approach, the ratio of the patient can be compared to both: a) the patient's own ratio over time; and b) a theoretical evolution of the ratio over time for people matching his condition (e.g., Alzheimer's disease or MCI), as defined by predictive algorithms. Consequently, two parameters can play a role: 1) evolution compared to others without treatment; and 2) evolution compared to the user's own ratio. This metric can be referred to as deviance from the curve, and can correspond to the effect of treatment. Turning to FIG. 7, depicted is ratio versus age for Alzheimer's disease patients not receiving treatment 701, for MCI patients not receiving treatment 703, for healthy people 705, and for Alzheimer's disease patients receiving treatment 707. Such treatment can be the sensory stimulation provided via VR headset discussed herein.

As reflected by FIG. 7, the discussed sensory stimulation provided via the VR headset can have a beneficial impact on the ratio. Compare, for instance, within FIG. 7 the line 701 for Alzheimer's disease patients not receiving sensory stimulation treatment versus the line 707 for Alzheimer's disease patients receiving sensory stimulation treatment. As is depicted by the figure, for at least patients of age circa 70 onward, the line 707 for Alzheimer's disease patients receiving sensory stimulation treatment is closer to line 705 for healthy people, than is the line 701 for patients not receiving sensory stimulation treatment.

The delivery of sensory stimulation (e.g., visual stimuli and auditory stimuli) can influence endogenous brainwaves via long-term potentiation neuroplasticity mechanisms. For instance, delivering stimulations in the theta and gamma ranges (e.g., at 4 and 40 hz combined) can have an impact on the evolution of the ratio compared to a patient not under treatment. As such, the evolution of this ratio can be used to provide patients, medical teams, and others with a progression score that directly reflects the effect of the provided sensory stimulation on a patient's electrical brain activity.

According to various embodiments, software of the system can perform various monitoring operations. These monitoring operations can, as just some examples, include monitoring device usability and monitoring hardware/firmware performance (e.g., to ensure stability and reliability of treatment). Based on this monitoring, operation of software of the system can be optimized and updated. In this way, benefits including increased therapeutical efficacy can accrue.

The system discussed herein is capable of being used at home by people suffering from neurodegenerative disorders, without a call for supervision by medical personnel. The software of the system can include instructions and tutorials to guide the patient on how to use the system (e.g., the VR headset thereof) and how to behave while stimulation is being delivered. Further, the software of the system can assess whether or not the patient follows provided usage instructions.

As just some examples, these usage instructions can: a) request that the patient sit in a quiet place when using the system; b) request that the patient use any prescribed corrective lenses (e.g., glasses) when using the system; and c) request that the patient perform various maintenance actions, such as charging the VR headset of the system between sessions.

By enforcing these usage instructions, the system can improve the functioning of software of the system, and can also enhance user interaction with the system. Enforcement by the system of the usage instructions can, as just some examples, utilize sensors integrated into the VR headset (e.g., patient-facing cameras) and analysis of various software, hardware, and firmware logs (e.g., in order to monitor battery levels during a given session). Further, enforcement by the system of the usage instructions can be implemented in adherence with applicable privacy regulations.

According to various embodiments, the system can be used as a combination therapy component. Since the pathogenesis of Alzheimer's disease is multifactorial, the use of a multimodal therapeutic intervention addressing several molecular targets of Alzheimer's disease-related pathological processes can be a useful approach to modify the course of disease progression. It has been demonstrated that the clinical efficacy of combination therapy can be higher than that of monotherapy.

Typically, combination therapy involves the use of multiple drugs. However, by including the system discussed herein a different sort of combination therapy can emerge. That is to say a combination therapy that on one hand utilizes one or more drugs, and on the other hand utilizes digital therapeutics in the form of the system discussed herein. Such integration of digital therapeutics has the potential to bolster drug assets throughout their entire lifecycle, from development to commercialization to post-marketing surveillance.

The synergistic effect of the sensory stimulation discussed herein and pharmacological intervention has the potential to not only enhance patient outcomes by targeting multiple targets, but to also: a) monitor disease progression (e.g., with neural and cognitive biomarkers); b) increase adherence by means of the VR environments discussed herein; and c) allow for the collection of real-world data to assess drug safety and to allow for decentralized clinical trials. The foregoing discussion of combination therapy involving the system discussed herein has generally focused on the treatment of Alzheimer's disease. However, it is noted that such combination therapy is also applicable to the treatment of other conditions.

The accumulation of Amyloid-B (Aβ) plaques and tau neurofibrillary tangles in the brain is a hallmark of Alzheimer's disease. In addition to this molecular pathology, disruption in amplitude or synchronization of gamma band oscillations is an additional contributor to cognitive decline in Alzheimer's disease.

Conventional approaches for treating Alzheimer's disease typically focus on reducing Aβ plaque buildup. However, the sensory stimulation approaches discussed herein offer an alternative approach with results that influence both neuroimmune mechanisms and gamma wave activity. The neuroimmune response has been measured in humans as the modulation of cytokines and immune factors in prodromal AD patients' CSF. Neural entrainment and subsequently increased gamma wave activity can be observed in humans at least in the visual cortex, in the pre-frontal cortex, and in deeper structures like the hippocampus. By eliciting a neuroimmune response and inducing neural entrainment, the sensory stimulation discussed herein offers a multifaceted approach that can yield a deceleration in neurodegeneration as measured in humans by, for example, reduced hippocampal atrophy, reduced ventricular enlargement, enhanced default mode network connectivity, improved sleep quality, and maintenance of daytime activities as assessed by the Alzheimer's Disease Cooperative Study-Activities of Daily Living Scale (ADCS-ADL) scale.

As such, integration of the sensory stimulation discussed herein with pharmacological interventions can provide a comprehensive approach that targets multiple molecular facets of Alzheimer's disease-related pathological processes, ultimately leading to enhanced patient outcomes. Accordingly, this holistic strategy can be beneficial in the pursuit of combating the devastating effects of Alzheimer's disease. Still, it is noted that the sensory stimulation discussed herein can prove beneficial to conditions including but not limited to Alzheimer's disease, even when applied alone.

The system described herein (including sensors such as EEG and ECG), along with the discussed cognitive tasks in VR, enable the identification of novel neural and cognitive biomarkers for early diagnosis of neurological disorders and ongoing disease monitoring. Such information can, as just some examples, be used to: a) evaluate or predict drug responses more effectively; and b) adjust drug dosages and treatment schedules so as to maximize therapeutic benefits, minimize side effects, and allow for optimal patient stratification. Turning to adherence, it has been found that individuals with cognitive impairments typically prefer VR environments over non-VR environments, as measured at least by improved mood and apathy in cohorts participating in immersive cognitive training and tasks. As such, the system discussed herein can promote active patient engagement through, as just some examples, immersive VR experiences, cognitive tasks, therapy guidance, and a connection to autobiographical memory through reminiscence. The interactive nature of the system can encourage patients to actively participate in their treatment, thereby promoting increased compliance with the sensory (e.g., audiovisual) stimulation, as well as with any medications that are also provided to the patient as part of a combination therapy approach. Further still, because the system is capable of being used at home and without a call for supervision by medical personnel, a patient using the system can experience a deepened sense of empowerment as to their health improvement.

Also, various capabilities of the system, including but not limited to the sensors thereof (e.g., EEG and ECG sensors) can allow the system to collect real-world evidence. The collected real-world evidence can be used in a multitude of ways, for instance in facilitating defense of safety claims regarding drug profiles. Further, the remote monitoring capabilities of the system offer benefits including but limited to allowing pharmaceutical companies the opportunity to decentralize clinical trials. Such decentralization is in alignment, for instance, with recent developments such as Medicare's decision to cover drugs based on real-world effectiveness data.

According to various embodiments, the VR headset can include an indicator light. The indicator light can serve to enhance the patient experience through the provision of interactions and of useful information. Further, in various embodiments the VR headset can be capable of connecting with a charging cradle, or of being directly plugged into an AC power source for recharging purposes.

The user interface of the VR headset can be manifested, at least in part, through the noted indicator light. The indicator light can be situated on the upper side of headband of the VR headset, as just an example. The indicator light can serve as a visual cue to convey diverse device statuses and interactions with the VR headset, thereby enriching the patient's engagement with the VR headset.

The indicator light, as just an example, be composed of a set of red, green, and blue (RGB) Light Emitting Diodes (LEDs). The LEDs can be harmoniously orchestrated to create the illusion of a continuous and uniform light band. This design choice can beneficially yield an indicator light is not only functional, but aesthetically pleasing as well.

To facilitate user interactions, the indicator light can be synchronized with, as just an example, either a proximity sensor or a touch sensor discretely positioned on the upper region of the headset. The proximity sensor can act to detect the presence of the patient, thereby enabling automatic device activation. When the VR headset is activated, the indicator light can also illuminate, thereby signaling the device's operational status to the patient.

In various embodiments, the indicator light can remain discreetly concealed within the housing of the VR headset when in an inactive state, thus preserving the sleek and unobtrusive appearance of the VR headset. Then, when the VR headset is activated, the indicator light can emit a discernible and informative glow through the housing. The indicator light can be used to communicate various device statuses to the user. Some example device statuses include:
Device Activation: When the VR headset is turned on, the indicator light can transition to a brilliant white hue and then gradually increases in luminance while creating an elegant and smooth linear fading effect, according to various embodiments.
Pairing Mode: For embodiments that support pairing, when the VR headset is ready to be paired with other devices the indicator light can transform into a serene blue shade, exhibiting a gentle and fading luminosity, as just an example. In this way, benefits including further enhancing the patient's understanding of the status of the VR headset can accrue.
Medical Alert (e.g., Seizure Detection): In various embodiments, in critical situations, such as the detection of a patient experiencing a seizure, the indicator light can shift to a prominent red color and initiate a pulsating pattern. In this way, the indicator light can promptly draw attention (e.g., the attention of a medical team member or of a caretaker), and convey the urgency of the situation.
Low Battery: To alert the patient to a low battery condition, the indicator light can transition to a vivid orange hue and employ an animated linear fading effect, drawing attention to the need for recharging, according to various embodiments.
Additional Device Status: Depending on specific use cases and requirements, the indicator light can be configured to communicate other device statuses and interactions as deemed necessary.

Where the VR headset incorporates an automatic shut-off feature, the indicator light can return to a serene white shade and gradually transition from a prominent linear effect to a smaller line, simultaneously decreasing in brightness until it eventually fades away completely, according to various embodiments. Further in this regard it is noted that, in various embodiments the VR headset can be equipped with auto-shut-off circuitry that activates when the headset is removed and placed on a surface. This circuitry can automatically power down the VR headset if it remains unused for an extended period (e.g., a 3-minute duration).

As such, the indicator light system of the VR headset can provide benefits including enhancing patient interaction and understanding of device statuses, and contributing to a more immersive and user-friendly VR experience.

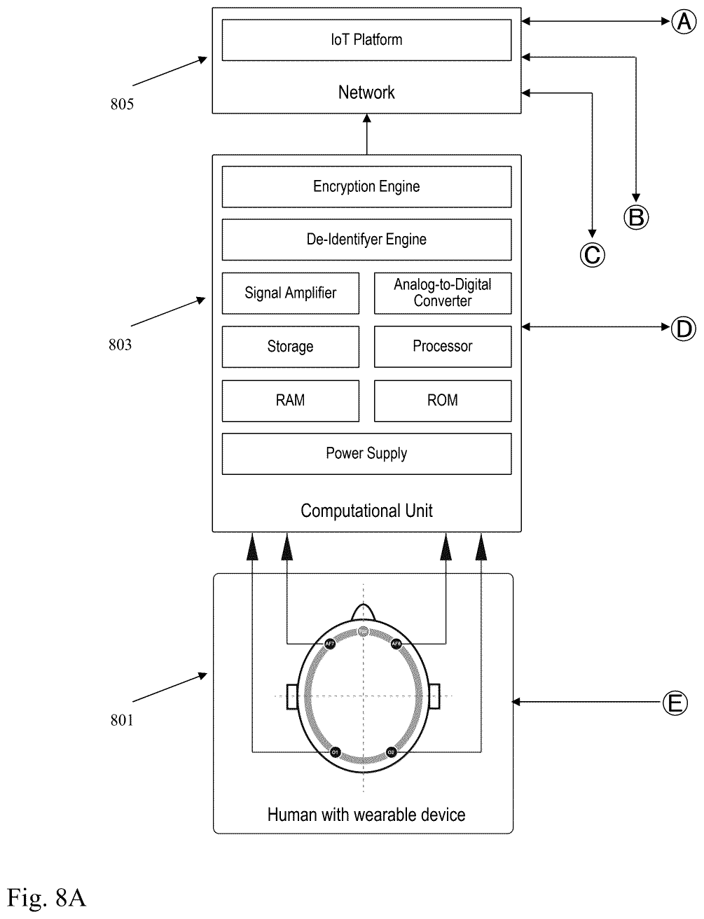

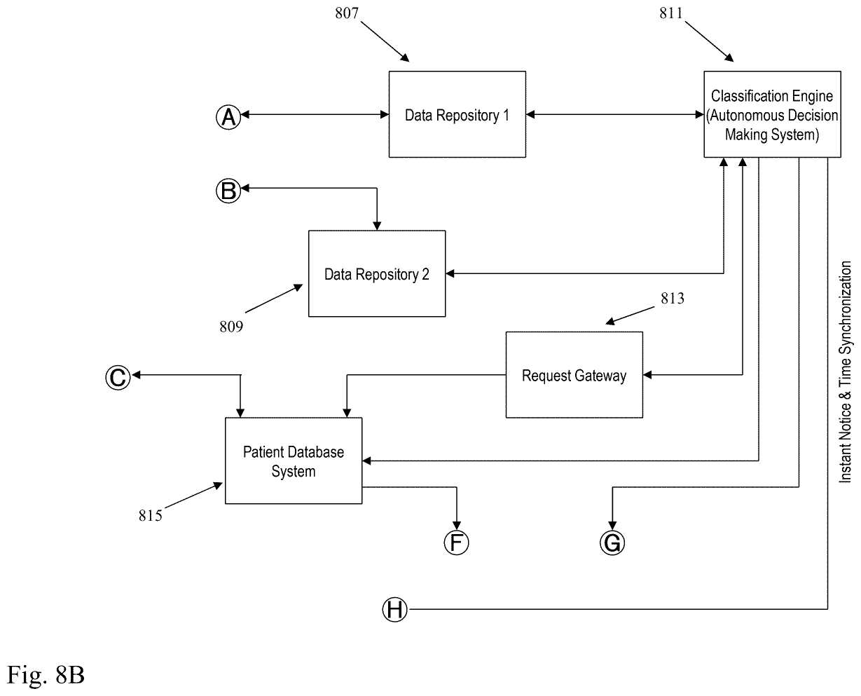

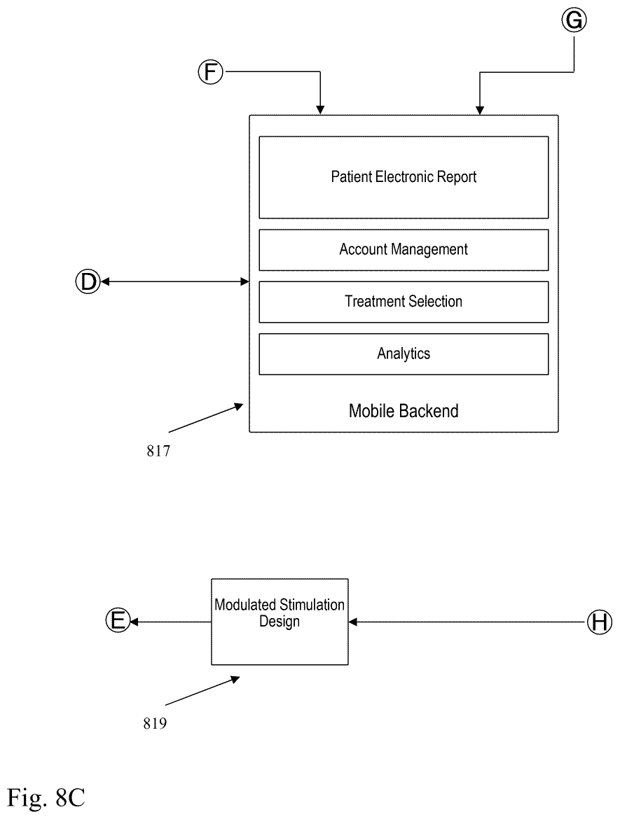

Shown in FIG. 8 an architectural diagram of an example implementation of the discussed system, according to various embodiments. According to the example of FIG. 8, the system can include a wearable device 801 (such as the discussed VR headset), a computational unit 803, a network unit 805, a first data repository 807, a second data repository 809, a classification/autonomous decision making system 811, a request gateway 813, a patient database system 815, a mobile backend 817, and a modulated simulation design unit 819.

The computational unit 803 can include an encryption engine, a de-identifier engine, a signal amplifier, and an analog-to-digital converter. In various embodiments, the computational unit 803 can also include a preprocessing module. The computational unit 803 can also include storage, one or more processors, random access memory (RAM), read-only memory (ROM), and power supply. The encryption engine can encrypt various data handled by the system, including patient-specific data. The de-identifier engine can anonymize various data handled by the system, including patient-specific data. The signal amplifier can amplify various of the signals discussed herein, such as EEG signals. The analog-to-digital converter can digitize various the signals discussed herein, such as EEG signals. The storage, processors, RAM, and ROM can be as discussed hereinbelow. The network unit 805 can support various communication capabilities discussed herein such as remote access by medical team members. The network unit 805 can include Internet of Things (IOT) capability, thereby allowing the system to act as an internet-connected data collection device and/or to be accessed via the internet. Here, such IoT capability can be implemented in a secure fashion.

The classification/autonomous decision making system 811 can perform various operations discussed herein including analyzing received physiological data (e.g., EEG data), and selecting stimulation protocols and/or VR scenarios. The modulated simulation design unit 819 can perform various operations discussed herein including interfacing with the wearable device 801 so as to implement stimulation protocols and/or VR scenarios selected by the classification/autonomous decision making system 811. Communication between the classification/autonomous decision making system 811 and the modulated simulation design unit 819 can, in various embodiments, involve instant notification and/or time synchronization, thereby supporting functionality including real time control of stimulation and the VR environment.

The mobile backend 817 can include patient electronic report capability, account management capability, treatment selection capability, and analytics capability. In various embodiments, the mobile backend 817 can also include signal processing capability. The mobile backend 817 can provide functionality including allowing medical team members to access reports regarding patient progress, manage patient accounts, make treatment selection choices, and access generated analytics data (e.g., health and disease progression scores of the sort discussed herein). The patient database system 815 can store various patient data discussed herein (e.g., patient progress reports and health scores). The first data repository 807 and second data repository 809 can store various data collected and generated by the system, such as collected sensor data (e.g., EEG data) and data corresponding to stimulation protocol and/or VR scenario selection and/or generation. The request gateway 813 perform various intermediary operations, including controlling access between the patient database system 815 and other system components (e.g., the classification/autonomous decision making system 811).

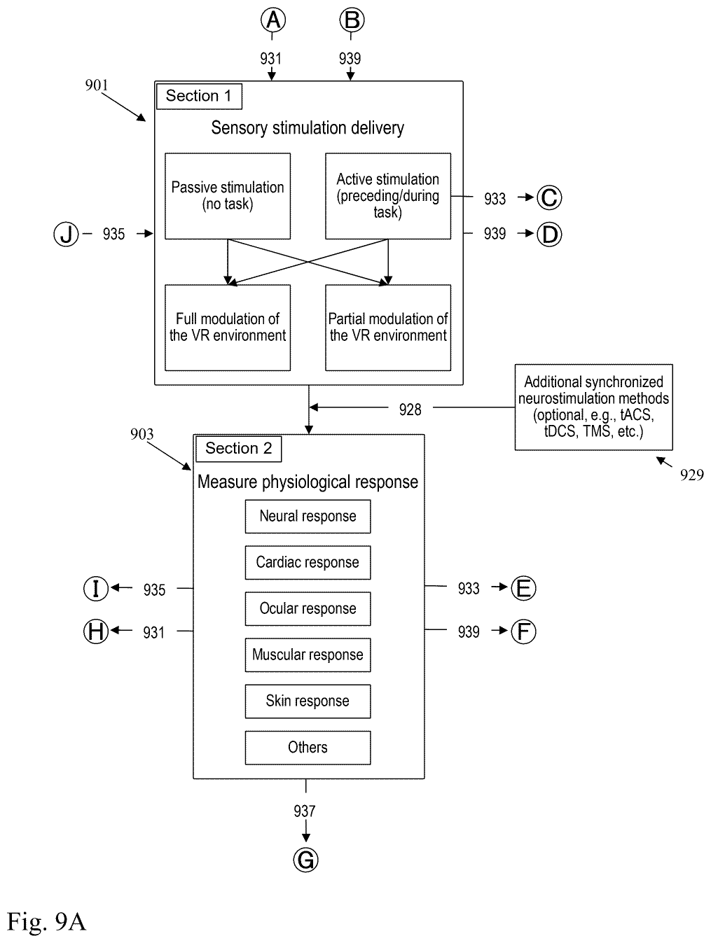

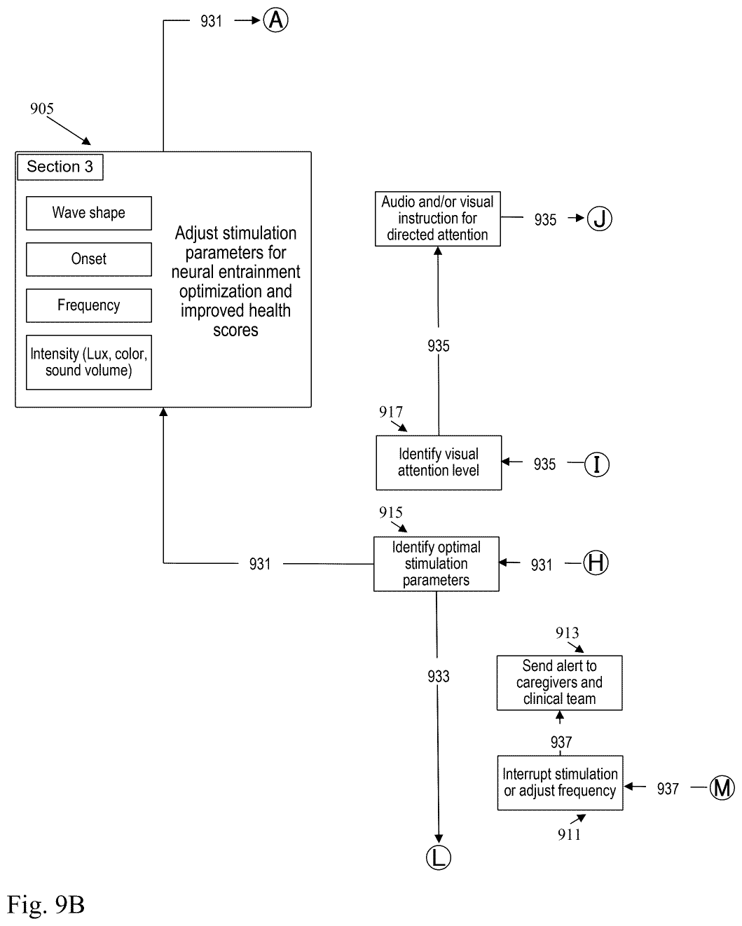

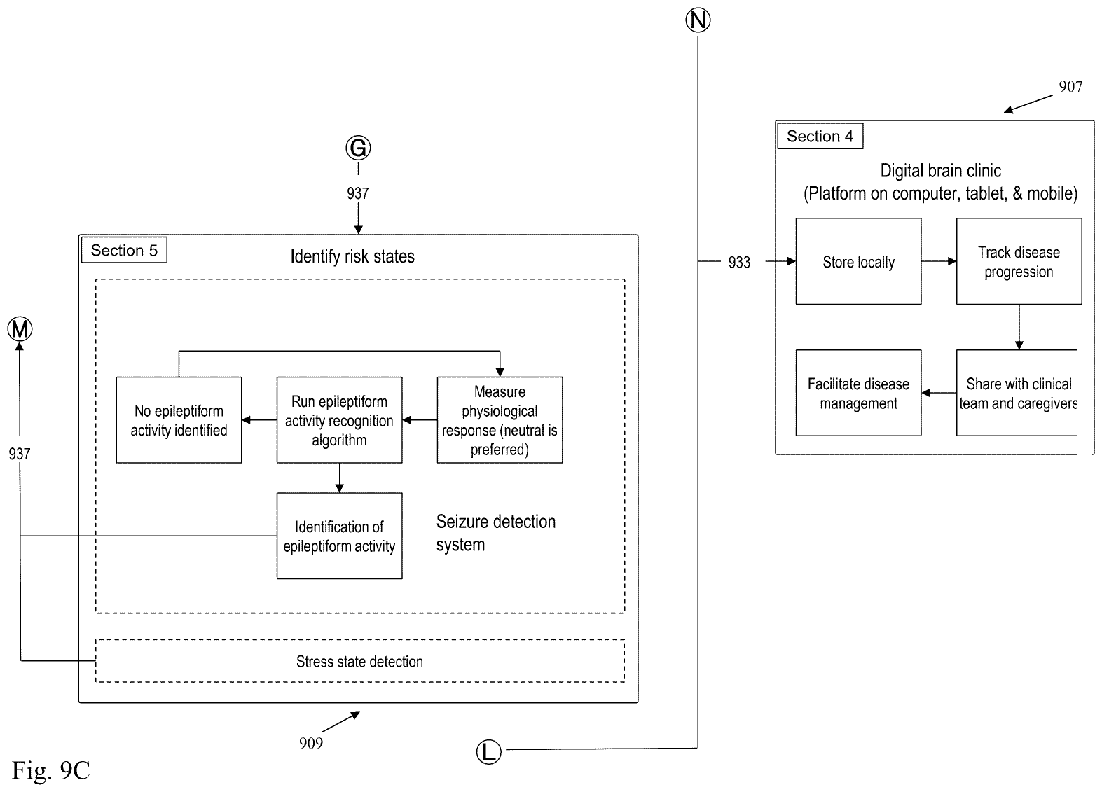

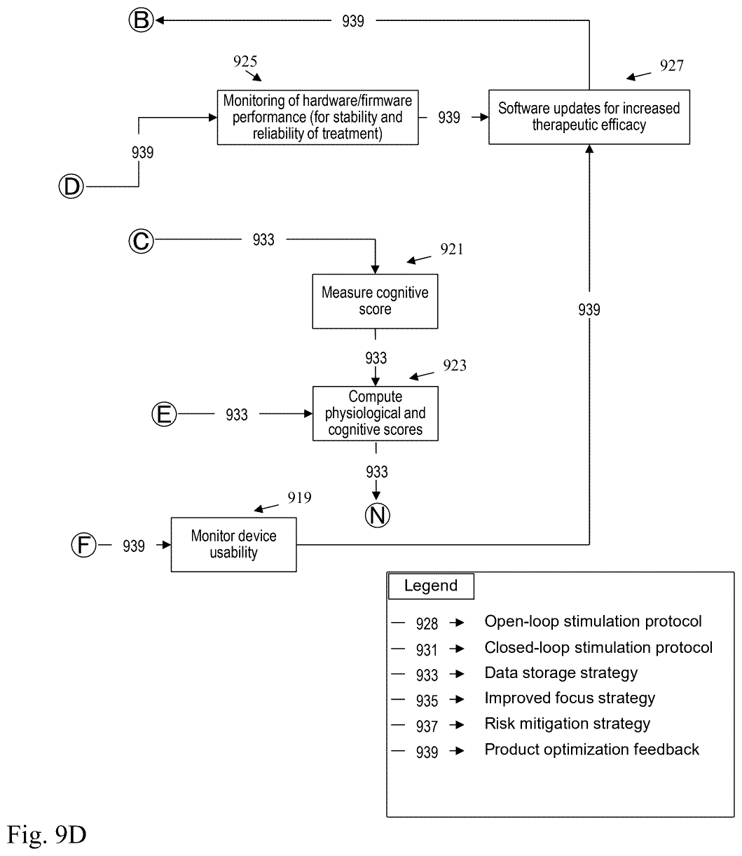

Turning to FIG. 9, is a schematic view depicting various capabilities of the system discussed herein. As shown by FIG. 9, the capabilities can include sensory stimulation delivery capabilities 901, physiological response measurement capabilities 903, stimulation adjustment capabilities 905, digital brain clinic capabilities 907, and risk state identification capabilities 909.

The sensory stimulation delivery capabilities 901 can include passive stimulation where no task is provided to the patient, and active stimulation where a task of the sort herein is assigned to the patient. Both of the passive stimulation and the active stimulation can involve partial modulation of the VR environment (e.g., where one or more VR objects flicker at the target frequency) or full modulation of the VR environment (e.g., where the entire VR environment flickers at the target frequency). The physiological response measurement capabilities 903 can include measuring neural response, cardiac response, ocular response, muscular response, and skin response. The stimulation adjustment capabilities 905 can include adjusting stimulation parameters, such as in order to optimize neural entrainment and to improve health scores. Such stimulation parameters can include wave shape, onset, frequency, and intensity (e.g., lux, color, and sound volume).

The digital brain clinic capabilities 907 can allow for the system to interface with devices such as personal computers, tablets, and smartphones. In particular, the digital brain clinic capabilities 907 can provide for access/sharing of patient data, tracking of disease progression, and management of disease treatment, such as by medical team members and caregivers. In various embodiments, the digital brain clinic capabilities 907 can provide for secure local storage of patient data at devices of the sort noted. The risk state identification capabilities 909 can include measuring patient physiological response (e.g., neural response) and subsequently running an epileptiform activity recognition algorithm. Based on the running of the epileptiform recognition algorithm, either epileptiform activity is identified, or epileptiform activity is not identified. Where epileptiform activity is identified, actions such as interrupting stimulation (or adjusting stimulation frequency) (911) and sending an alert to caregivers and medical team members can be taken (913). The risk state identification capabilities 909 can also implement stress state alert functionality in accordance with that which is discussed above.

With further reference to FIG. 9, and with further reference that which is discussed hereinabove, further operations can include identification of optimal stimulation parameters (915), and identification of visual attention level (917). With additional reference to FIG. 9, and with further reference that which is discussed hereinabove, operations can also include monitoring device usability (919), performing cognitive and physiological measurements (921), and computing cognitive and physiological scores (923). With still further reference to FIG. 9, and with reference that which is discussed hereinabove, operations can also include monitoring hardware/firmware performance (e.g., to ensure stability and reliability of treatment) (925) and optimizing/updating software of the system (e.g., to ensure stability and reliability of treatment) (927). Further with reference to FIG. 9, in various embodiments beyond sensory stimulation, additional synchronized neurostimulation techniques can be provided to the patient (929). These additional techniques can include tACS, tDCS, and TMS.

Also with reference to FIG. 9, it is noted that functionality of the system can include open-loop stimulation protocol functionality (928), closed-loop stimulation protocol functionality (931), data storage strategy functionality (933), improved focus strategy functionality (935), risk mitigation strategy functionality (937), and product optimization feedback functionality (939).

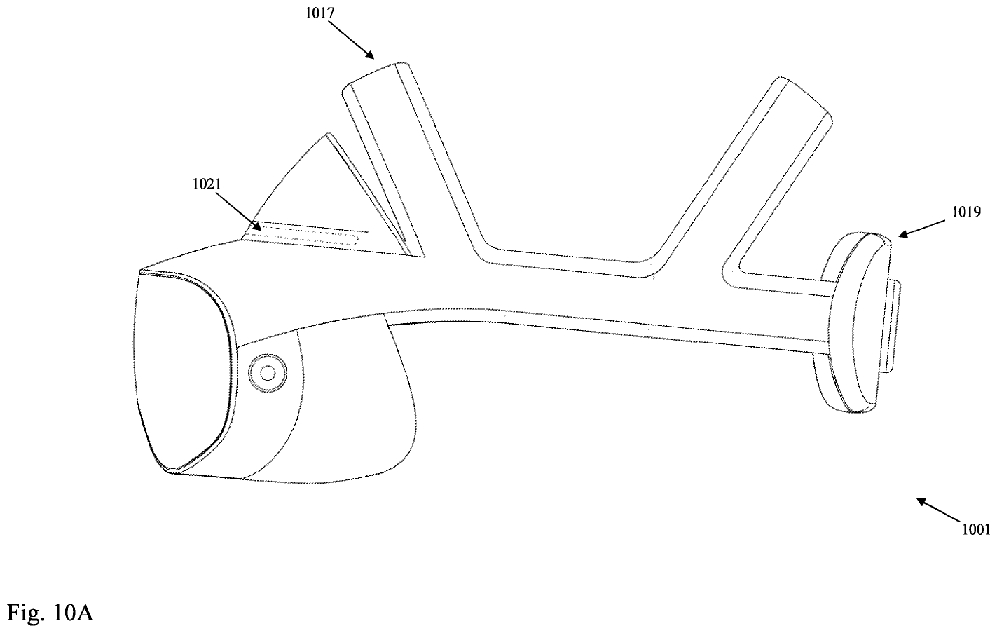

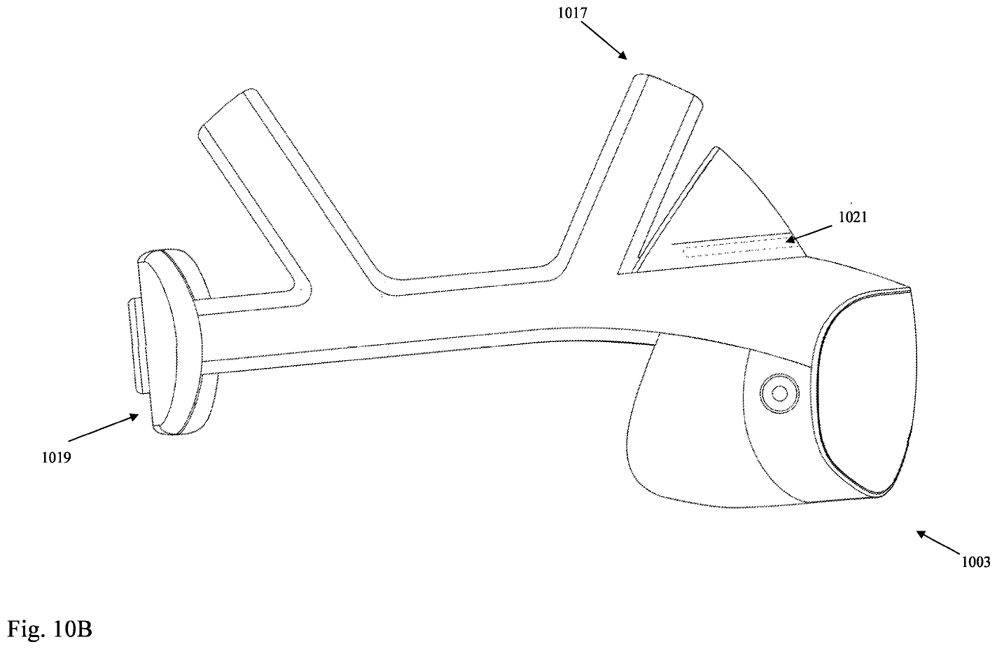

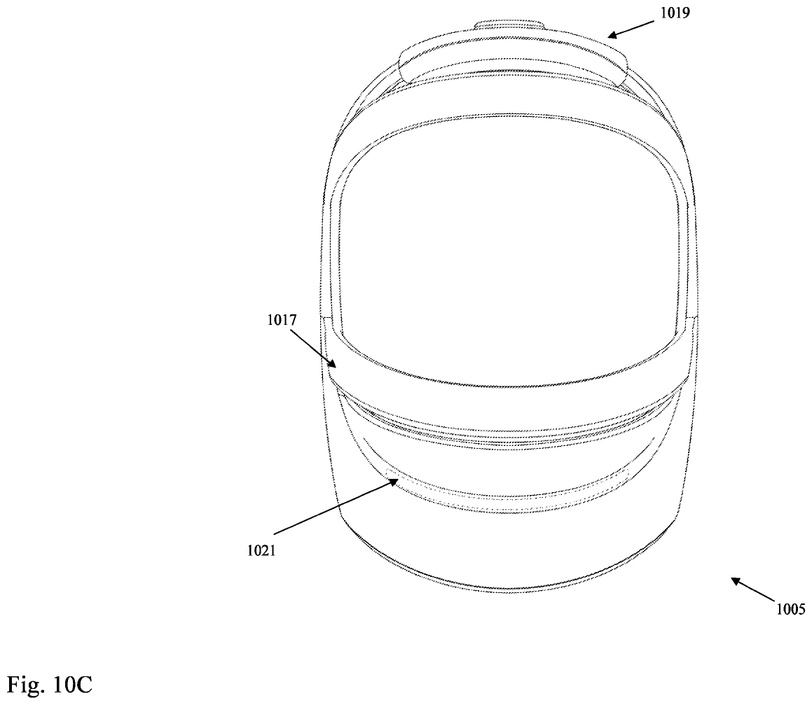

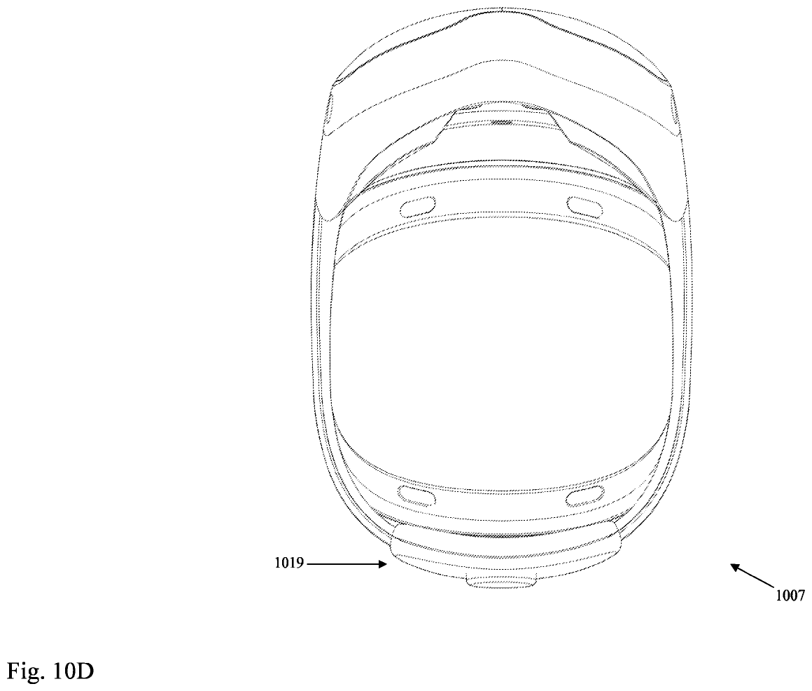

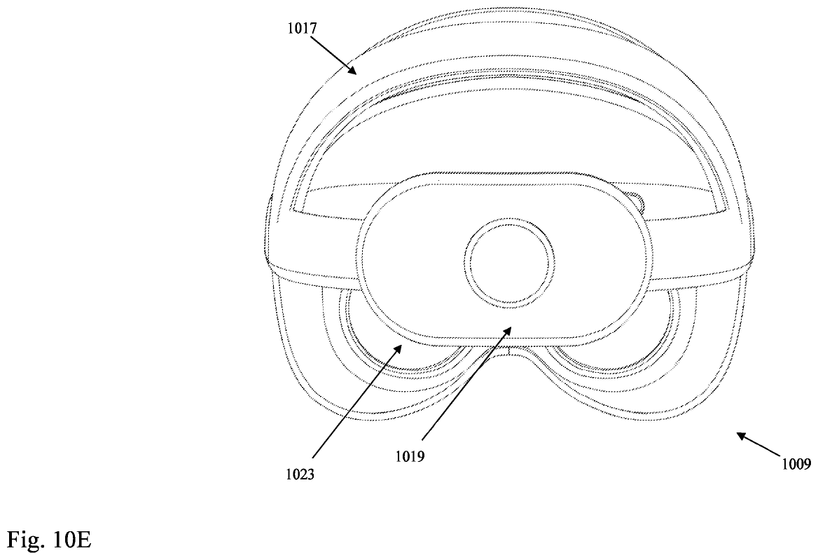

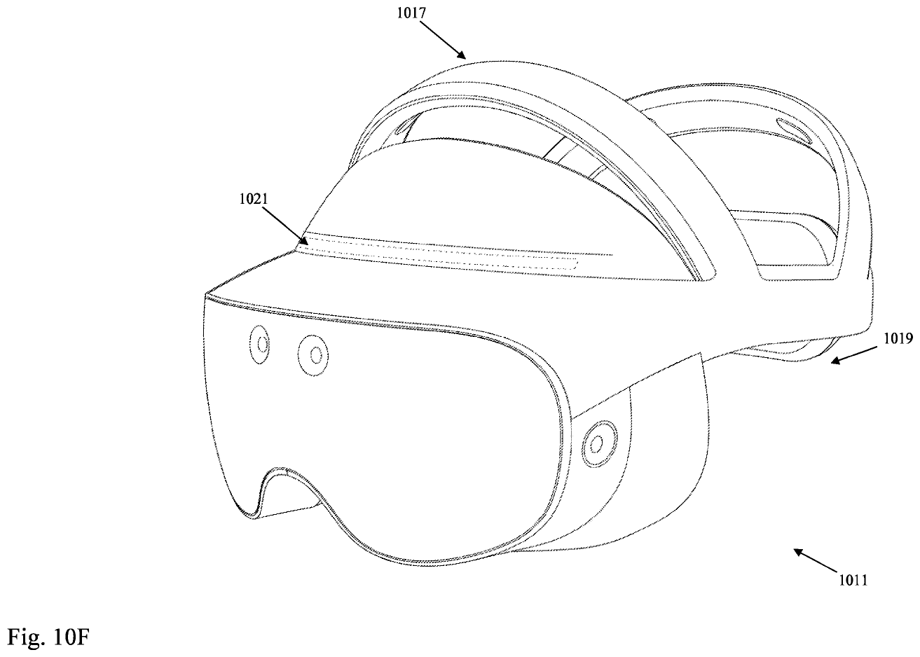

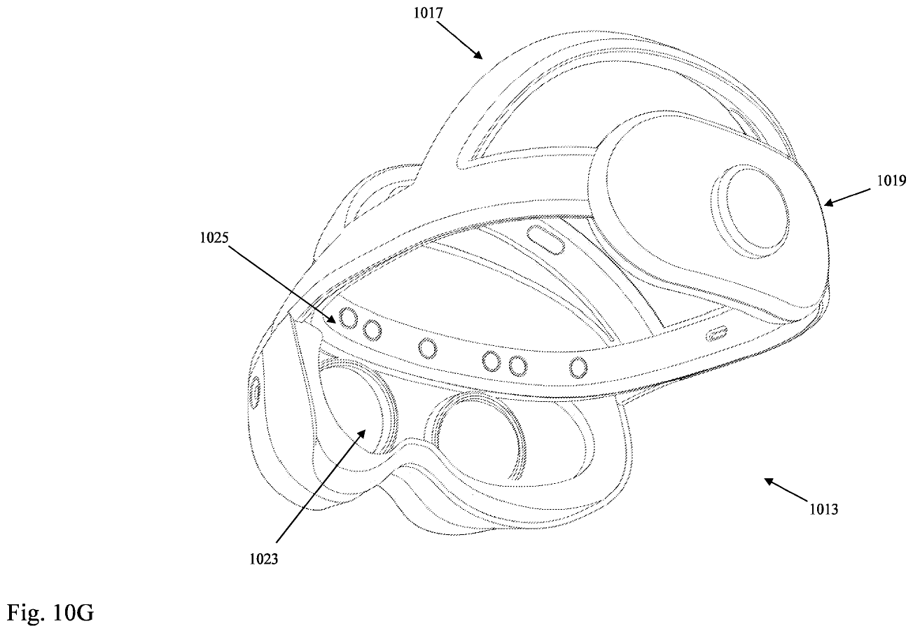

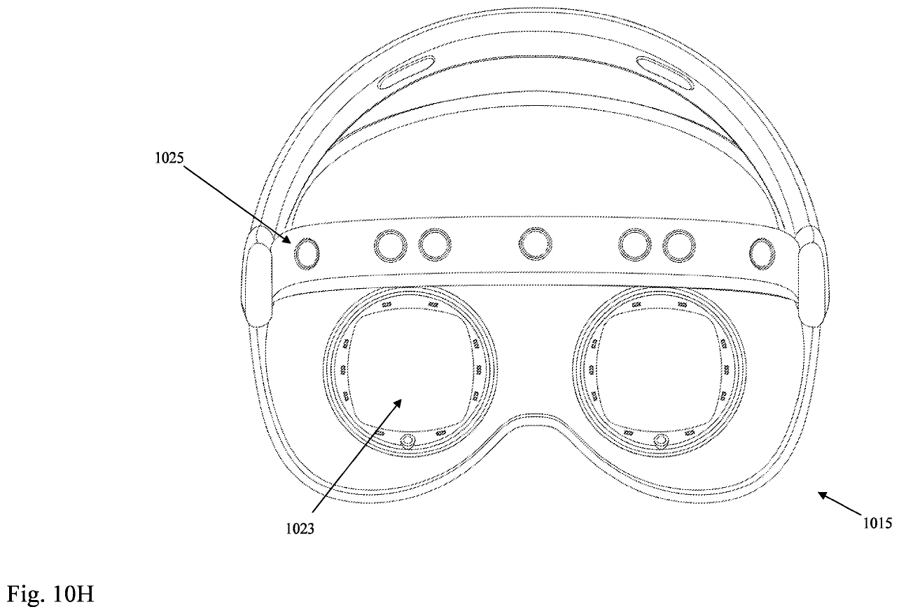

Turning to FIGS. 10A-10H, shown are various views of an example implementation of the VR headset according to various embodiments. In particular, FIG. 10A depicts a left side view 1001 of the VR headset, FIG. 10B depicts a right side view 1003 of the VR headset, FIG. 10C depicts a top view 1005 of the VR headset, FIG. 10D depicts a bottom view 1007 of the VR headset, and FIG. 10E depicts a back view 1009 of the VR headset. Further, FIG. 10F depicts an outside perspective view 1011 of the VR headset, FIG. 10G depicts an inside perspective view 1013 of the VR headset, and FIG. 10H depicts a back view 1015. of the VR headset. Also depicted by these figures are various of the VR headset elements including headband 1017, headband adjustor 1019, indicator light 1021, screen 1023, and sensors (e.g., EEG sensors) 1025.

### Hardware and Software

According to various embodiments, various functionality discussed herein can be performed by and/or with the help of one or more computers. Such a computer can be and/or incorporate, as just some examples, a personal computer, a server, a smartphone, a system-on-a-chip, and/or a microcontroller. Such a computer can, in various embodiments, run Linux, MacOS, Windows, or another operating system.

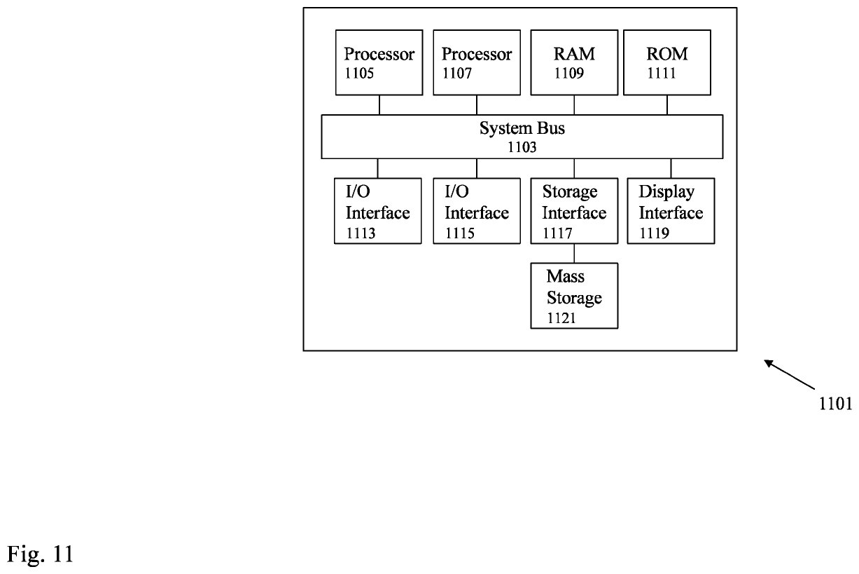

Such a computer can also be and/or incorporate one or more processors operatively connected to one or more memory or storage units, wherein the memory or storage may contain data, algorithms, and/or program code, and the processor or processors may execute the program code and/or manipulate the program code, data, and/or algorithms. Shown in FIG. 11 is an example computer employable in various embodiments of the present invention. Example computer 1101 includes system bus 1103 which operatively connects two processors 1105 and 1107, random access memory (RAM) 1109, read-only memory (ROM) 1111, input output (I/O) interfaces 1113 and 1115, storage interface 1117, and display interface 1119. Storage interface 1117 in turn connects to mass storage 1121. Each of I/O interfaces 1113 and 1115 can, as just some examples, be a Universal Serial Bus (USB), a Thunderbolt, an Ethernet, a Bluetooth, a Long Term Evolution (LTE), a 5G, an IEEE 488, and/or other interface. Mass storage 1121 can be a flash drive, a hard drive, an optical drive, or a memory chip, as just some possibilities.

Processors 1105 and 1107 can each be, as just some examples, a commonly known processor such as an ARM-based or x86-based processor. Computer 1101 can, in various embodiments, include or be connected to a touch screen, a mouse, and/or a keyboard. Computer 1101 can additionally include or be attached to card readers, DVD drives, floppy disk drives, hard drives, memory cards, ROM, and/or the like whereby media containing program code (e.g., for performing various operations and/or the like described herein) may be inserted for the purpose of loading the code onto the computer.

In accordance with various embodiments of the present invention, a computer may run one or more software modules designed to perform one or more of the above-described operations. Such modules can, for example, be programmed using Python, Java, Javascript, Swift, C, C++, C#, and/or another language. Corresponding program code can be placed on media such as, for example, DVD, CD-ROM, memory card, and/or floppy disk. It is noted that any indicated division of operations among particular software modules is for purposes of illustration, and that alternate divisions of operation may be employed. Accordingly, any operations indicated as being performed by one software module can instead be performed by a plurality of software modules. Similarly, any operations indicated as being performed by a plurality of modules can instead be performed by a single module. It is noted that operations indicated as being performed by a particular computer can instead be performed by a plurality of computers. It is further noted that, in various embodiments, peer-to-peer and/or grid computing techniques may be employed. It is additionally noted that, in various embodiments, remote communication among software modules may occur. Such remote communication can, for example, involve JavaScript Object Notation-Remote Procedure Call (JSON-RPC), Simple Object Access Protocol (SOAP), Java Messaging Service (JMS), Remote Method Invocation (RMI), Remote Procedure Call (RPC), sockets, and/or pipes.

Moreover, in various embodiments the functionality discussed herein can be implemented using special-purpose circuitry, such as via one or more integrated circuits, Application Specific Integrated Circuits (ASICs), or Field Programmable Gate Arrays (FPGAs). A Hardware Description Language (HDL) can, in various embodiments, be employed in instantiating the functionality discussed herein. Such an HDL can, as just some examples, be Verilog or Very High Speed Integrated Circuit Hardware Description Language (VHDL). More generally, various embodiments can be implemented using hardwired circuitry without or without software instructions. As such, the functionality discussed herein is limited neither to any specific combination of hardware circuitry and software, nor to any particular source for the instructions executed by the data processing system.

1. A computer-implemented method, comprising:
presenting, by a computing system via a virtual reality headset, a stimulation protocol;
presenting, by the computing system via the virtual reality headset, a virtual reality scenario;
receiving, by the computing system from one or more sensors, patient physiological data; and
adapting, by the computing system using the patient physiological data, one or more of the stimulation protocol or the virtual reality scenario.

2. The computer-implemented method of claim 1, wherein the patient physiological data includes patient brain activity data.

3. The computer-implemented method of claim 1, wherein the presented stimulation protocol includes one or more of visual stimuli or auditory stimuli.

4. The computer-implemented method of claim 1, wherein the presented stimulation protocol includes one or more of 4 Hz stimuli, 40 Hz stimuli, combined 4 Hz/40 Hz stimuli, beta-range stimuli, or alpha-range stimuli.

5. The computer-implemented method of claim 1, further comprising:
receiving, by the computing system from one or more sensors, patient brain activity data collected during patient performance of a task that elicits a brainwave state of interest; and
determining, by the computer system, from the received brain activity data, a patient-specific frequency value corresponding to the brainwave state of interest.

6. The computer-implemented method of claim 5, wherein the brainwave state of interest is beta, alpha, theta, or gamma.

7. The computer-implemented method of claim 1, further comprising:
detecting, by the computing system from the received patient physiological data, abnormal neural activity; and
stopping, by the computing system, one or more of the presented stimulation protocol or the presented virtual reality scenario.

8. The computer-implemented method of claim 1, wherein the presented virtual reality scenario includes one or more of a cognitive task, a functional task, or a gamified task.

9. The computer-implemented method of claim 8, further comprising:
determining, by the computing system, a score based on patient performance of said task.

10. The computer-implemented method of claim 1, wherein the presentation of the stimulation protocol and the presentation of the virtual reality scenario are performed as a combination therapy component along with application of one or more pharmaceuticals.

11. The computer-implemented method of claim 1, further comprising:
determining, by the computing system, using an eye tracking system, whether patient attention is directed to visual stimuli of the presented virtual reality scenario; and
presenting, by the computing system via the virtual reality headset, patient feedback regarding said attention determination.

12. The computer-implemented method of claim 1, wherein the stimulation protocol includes visual stimuli, and wherein said presentation of the stimulation protocol includes one or more of:
modulating presentation within a VR environment of displayed content;
modulating presentation within a VR environment of a frame, wherein the frame is situated around displayed content; or
modulating presentation of at least one object within a VR environment, wherein said modulation is at a target frequency.

13. The computer-implemented method of claim 1, further comprising:
providing, by the computing system, remote access, wherein the remote access allows for one or more of access to patient progress reports or selection of patient treatment options.

14. A system comprising:
at least one processor; and
a memory storing instructions that, when executed by the at least one processor, cause the system to perform the computer-implemented method of claim 1.

15. A non-transitory computer-readable storage medium including instructions that, when executed by at least one processor of a computing system, cause the computing system to perform the computer-implemented method of claim 1.
